(19) United States
(12) Patent Application Publication
(10) Pub. No.: US 2013/0095121 A1
(43) Pub. Date: Apr. 18, 2013

(54) METHODS OF TREATING PATIENTS WITH IMMUNE-RELATED DISEASES

(71) Applicants: Aoife Brennan, Dover, MA (US); Charlotte McKee, Newton, MA (US); Lou Vaickus, Hingham, MA (US)

(72) Inventors: Aoife Brennan, Dover, MA (US); Charlotte McKee, Newton, MA (US); Lou Vaickus, Hingham, MA (US)

(21) Appl. No.: 13/704,692

(22) PCT Filed: Jun. 24, 2011
(86) PCT No.: PCT/US11/41777

§ 371 Date: Dec. 17, 2012

Related U.S. Application Data
(60) Provisional application No. 61/358,736, filed on Jun. 25, 2010.

(51) Int. Cl. A61K 39/395

(52) U.S. Cl. CPC A61K 39/3955
USPC 424/173.1

(57) ABSTRACT

Provided herein are methods of treating patients with immune-related diseases (e.g., diabetes) with immunotherapy.

### TECHNICAL FIELD

This document relates to methods for treating patients with immune-related diseases, and more particularly to using immunotherapy to treat patients with immune-related diseases if at least one marker of the immune-related disease and at least one marker of pathogenic immunological activity are identified in the patient.

### BACKGROUND

Immune-related diseases and their complications are major causes of morbidity and mortality in the United States, and substantially contribute to health-care costs. For diabetes alone, it is estimated that 23.6 million people have diagnosed or undiagnosed diabetes, leading to over $174 billion in direct and indirect health care costs. See CDC National Diabetes Fact Sheet, 2007. Thus, there is a need for methods of treating immune-related disease.

### SUMMARY

This document provides methods of treating immune-related diseases (e.g. diabetes) in patients in which one or more markers of the immune-related disease and one or more markers of pathogenic immunological (e.g., T cell) activity have been identified.

In one aspect, this document features a method of treating a patient suspected of having an immune-related disease. The method includes (a) identifying a biomarker of the immune-related disease in the patient; (b) identifying a marker of pathogenic immunological activity in the patient; and (c) treating the patient with a therapeutically effective course of immunotherapy if the biomarker of the immune-related disease and the marker of pathogenic immunological activity are identified in the patient.

This document also features a method of treating a patient suspected of having an immune-related disease. The method includes (a) identifying a biomarker of the immune-related disease in the patient; (b) identifying a marker of pathogenic immunological activity in the patient; and (c) treating the patient with a therapeutically effective course of treatment if the biomarker of the immune-related disease and the marker of pathogenic immunological activity are identified in the patient. The course of treatment can include a dosing regimen with an anti-CD3 antibody or antigen-binding fragment thereof, wherein the antibody or fragment does not bind or has reduced binding to at least one Fc (gamma) receptor compared to the IgG1 immunoglobulin molecule produced by the cell line ARH-77 deposited under ATCC catalog number CRL-1621.

This document also features a method of treating a patient suspected of having diabetes. The method includes (a) identifying a biomarker of diabetes in the patient; (b) identifying a marker of pathogenic immunological activity in the patient; and (c) treating the patient with a therapeutically effective course of immunotherapy if the marker of diabetes and the marker of pathogenic immunological activity are identified in the patient.

In another aspect, this document features a method of treating a patient suspected of having diabetes. The method includes (a) identifying a biomarker of diabetes in the patient; (b) identifying a marker of pathogenic immunological activity in the patient; and (c) treating the patient with a therapeutically effective course of treatment if the marker of the immune-related disease and the marker of pathogenic immunological activity are identified in the patient. The course of treatment can include a dosing regimen with an anti-CD3 antibody or antigen-binding fragment thereof, wherein the antibody or fragment does not bind or has reduced binding to at least one Fc (gamma) receptor compared to the IgG1 immunoglobulin molecule produced by the cell line ARH-77 deposited under ATCC catalog number CRL-1621.

This document also features a method of re-treating a patient with an immune-related disease, wherein the patient has undergone at least one course of treatment with immunotherapy after identification in the patient of a biomarker of the immune-related disease and a marker of pathogenic immunological activity. The method includes a) monitoring the patient for an indicator of return to active disease; and b) re-dosing the patient with an additional course of treatment with immunotherapy if the indicator is identified in the patient.

This document also features a method of re-treating a patient with an immune-related disease, wherein the patient has undergone at least one course of treatment with a dosing regimen with an anti-CD3 antibody or antigen-binding fragment thereof after identification in the patient of a biomarker of the immune-related disease and a marker of pathogenic immunological activity, and wherein the antibody or fragment does not bind or has reduced binding to at least one Fc (gamma) receptor compared to the IgG1 immunoglobulin molecule produced by the cell line ARH-77 deposited under ATCC catalog number CRL-1621. The method includes monitoring the patient for an indicator of return to active disease; and re-dosing the patient with an additional course of treatment with a dosing regimen of the anti-CD3 antibody or antigen-binding fragment thereof if the indicator is identified in the patient.

In another aspect, this document features a method of re-treating a patient with diabetes, wherein the patient has undergone at least one course of treatment with immunotherapy after identification in the patient of a biomarker of diabetes and a marker of pathogenic immunological activity. The method includes monitoring the patient for an indicator of return to active disease; and re-dosing the patient with an additional course of treatment with immunotherapy if the indicator is identified in thepatient.

This document also features a method of re-treating a patient with diabetes, wherein the patient has undergone at least one course of treatment with a dosing regimen with an anti-CD3 antibody or antigen-binding fragment thereof after identification in the patient of a biomarker of diabetes and a marker of pathogenic immunological activity, and wherein the antibody or fragment does not bind or has reduced binding to at least one Fc (gamma) receptor compared to the IgG1 immunoglobulin molecule produced by the cell line ARH-77 deposited under ATCC catalog number CRL-1621. The method includes monitoring the patient for an indicator of return to active disease; and re-dosing the patient with an additional course of treatment with a dosing regimen of the anti-CD3 antibody or fragment thereof if the indicator is identified in the patient.

Methods described herein can include measuring blood glucose variability as a factor in determining if a patient has an increased risk of long and short-term complications of diabetes.

This document also features diagnostic tests and assays for identifying biomarkers of immune-related diseases such as diabetes and diagnostic tests and assays for identifying markers of pathogenic immunological activity. Each such test or assay can be used individually or in combination, or individually or in combination with the methods described herein for treating patients with immune-related diseases or for selecting patients for which immunotherapy is a suitable method of treatment.

In any of the methods, diagnostic tests, or assays described herein, the marker of pathogenic immunological activity can be pathogenic T cell activity or autoimmune activity (e.g., autoimmune T cell activity). A marker of autoimmune activity can be selected from the group consisting of autoantibodies, autoantigen-responsive T cells, or autoreactive T cells expressing particular autoantigen-specific T cell receptors. For example, the marker of autoimmune activity can be the presence of an autoantibody such as an anti-glutamic acid decarboxylase autoantibody, anti-protein tyrosine phosphatase-like protein autoantibody (anti-IA-2), anti-zinc transporter autoantibody, and insulin autoantibody.

In any of the methods, diagnostic tests, or assays described herein, the marker of pathogenic immunological activity can be an abnormal level of one or more cytokines.

In the methods, diagnostic tests, or assays described herein, the patient can have an immune-related disease (e.g., an autoimmune disease). In some embodiments, the immune-related disease is diabetes (e.g., type 1 diabetes). In some embodiments, the immune-related disease is selected from the group consisting of Crohn's disease, Graves' disease, Graves' ophthalmopathy, lupus erythematosus, multiple sclerosis, myasthenia gravis, psoriasis, psoriatic arthritis, rheumatoid arthritis, Sjögren's syndrome, systemic lupus erythematosus, and ulcerative colitis.

In any of the methods, diagnostic tests, or assays described herein, the biomarker of the immune-related disease or the biomarker of diabetes can be a biomarker of beta cell destruction (e.g., amylin, glucagon, an islet-associated protein, insulin production, glucose tolerance, glucose variability, insulin dose-adjusted HbA1c, or HbA1c). For example, in some embodiments, the biomarker of beta cell destruction is insulin production. Detecting insulin production can include determining blood or urine level of C-peptide.

In any of the methods, diagnostic tests, or assays described herein, the biomarker of the immune-related disease or the biomarker of diabetes can be pancreatic islet inflammation.

In some embodiments, the immune-related disease is rheumatoid arthritis and the biomarker of rheumatoid arthritis is selected from the group consisting of follistatin-like-protein-1, C-reactive protein (CRP), erythrocyte sedimentation rate (ESR), anti-CCP antibody, serum amyloid A, rheumatoid factor, IL-6, 5100, osteopontin, MMP-1, MMP-3, hyaluronic acid, and a product of collagen metabolism.

In some embodiments, the immune-related disease is Graves' Disease and the biomarker of Graves' Disease is soluble CTLA-4.

In some embodiments, the immune-related disease is psoriasis and the biomarker of psoriasis is platelet P-selectin or soluble P-selectin.

In any of the methods described herein, an indicator of return to active disease can be selected from the group consisting of increased insulin requirements; a change of HbA1c; a change of insulin dose-adjusted HbA1c, a change in fasting C-peptide; a change in glucose tolerance tests; increased incidence of abnormal glucose measurements; detection of autoantibodies against one or more islet cell antigens; detection of islet cell antigen specific T cells; a decrease in beta-cell mass; and increased incidence of hypoglycemic or ketoacidosis episodes.

In any of the methods described herein, immunotherapy can include (a) administration of an antibody, or antigen binding fragment thereof, that binds to an immunomodulatory molecule on the surface of an immune cell, or (b) administration of an agent comprising a soluble ligand or receptor, or a functional fragment thereof, that binds to an immunomodulatory molecule on the surface of an immune cell. The immunomodulatory molecule can be a co-stimulatory molecule or a receptor for a co-stimulatory molecule. The immunomodulatory molecule can be selected from the group consisting a molecule of the CD3 complex, a T cell receptor molecule, a CD4 molecule, a CD8 molecule, a CD20 molecule, a B7 molecule, and a 4-1BB molecule. The antibody or antigen binding fragment thereof can be an agonistic antibody or fragment thereof or can be an antagonistic antibody or fragment thereof.

In any of the methods described herein, immunotherapy can include a course of treatment with a dosing regimen of an anti-CD3 antibody or antigen-binding fragment thereof, wherein the anti-CD3 antibody or fragment thereof does not bind or has reduced binding to at least one Fc (gamma) receptor compared to the IgG1 immunoglobulin molecule produced by the cell line ARH-77 deposited under ATCC catalog number CRL-1621, and wherein over the course of treatment, the total amount of the anti-CD3 antibody or fragment thereof administered to the patient does not exceed 300 μg/kg when administered intravenously, and when administered other than intravenously, the total amount administered does not exceed the bioequivalent of intravenous administration of 300 μg/kg.

In any of the methods described herein, an additional course of treatment can include a dosing regimen of an anti-CD3 antibody or antigen-binding fragment thereof, wherein the anti-CD3 antibody or fragment does not bind or has reduced binding to at least one Fc (gamma) receptor compared to the IgG1 immunoglobulin molecule produced by the cell line ARH-77 deposited under ATCC catalog number CRL-1621, and wherein over the course of treatment, the total amount of the anti-CD3 antibody or fragment thereof administered to said patient does not exceed 300 μg/kg when administered intravenously, and when administered other than intravenously, the total amount administered does not exceed the bioequivalent of intravenous administration of 300 μg/kg.

In any of the methods described herein, the anti-CD3 antibody antigen-binding fragment can be selected from the group consisting of a Fab fragment, a F(ab′)2 fragment and a scFv fragment. In any of the methods described herein, the anti-CD3 antibody or fragment can be chimeric or can be humanized. In any of the methods described herein, the antibody or fragment can include an Fc domain, wherein the Fc domain is aglycosylated. In any of the methods described herein, the antibody or fragment can include the amino acid sequence of SEQ ID NO: 3. In any of the methods described herein, the antibody or fragment can include the amino acid sequence of SEQ ID NO: 4. In any of the methods described herein, the antibody or fragment can include SEQ ID NO: 3, and further include SEQ ID NO: 4. In any of the methods described herein, the antibody or fragment can have an alanine at an amino acid position corresponding to amino acid position 299 of SEQ ID NO: 1. In any of the methods described herein, the antibody can be hOKT3, hOKT3γ1(Ala-Ala), HUM291, NI-0401. In any of the methods described herein, the antibody or fragment can exhibit at least 50% reduced binding to at least one Fc (gamma) receptor compared to the IgG1 antibody deposited under ATCC accession number CRL-1621. In any of the methods described herein, the antibody or fragment can exhibit at least 50% reduced binding to at least one Fc (gamma) receptor compared to the OKT3 antibody.

In any of the methods described herein, the dosage regimen can include administering doses of increasing amounts of the antibody or fragment thereof on at least the initial three days of the dosage regimen.

In any of the methods described herein, the dosing regimen can include five or more days of dosing (e.g., eight days of dosing).

In any of the methods described herein, the dosing regimen can be at least five days of dosing; wherein the antibody or fragment is administered on day one, and wherein the amount of antibody or fragment administered on each of days one and two does not exceed 0.5 mg per day; wherein the amount of antibody or fragment administered on day three is less than about 0.5 mg greater than the amount of antibody or fragment administered on day two; wherein the amount of antibody or fragment administered on day four is less than about 0.55 mg greater than the amount of antibody or fragment administered on day three; wherein the amount of antibody or fragment administered on day five is less than about 0.6 mg greater than the amount of antibody or fragment administered on day four; wherein the amount of antibody or fragment administered on day five is more than 0.3 mg greater than the amount of antibody or fragment administered on day two; and wherein the amount of antibody or fragment administered on day five is at least about 0.5 mg.

In any of the methods described herein, the antibody or fragment can be administered over a dosing regimen comprising at least four ramp days; wherein the antibody or fragment is administered in an amount greater than about 0.1 mg and less than about 0.5 mg on ramp day one; wherein the amount of antibody or fragment administered on ramp day two is less than about 0.5 mg greater than the amount of antibody or fragment administered on ramp day one; wherein the amount of antibody or fragment administered on ramp day three is less than about 0.55 mg greater than the amount of antibody or fragment administered on ramp day two; wherein the amount of antibody or fragment administered on ramp day four is less than about 0.6 mg greater than the amount of antibody or fragment administered on ramp day three; wherein the amount of antibody or fragment administered on ramp day four is more than 0.3 mg greater than the amount of antibody or fragment administered on ramp day one; and wherein the amount of antibody or fragment administered at least one ramp day is at least about 0.5 mg. The antibody or fragment can be administered on at least one pre-ramp day prior to ramp day one.

In any of the methods described herein, the antibody or fragment can be administered intravenously. In any of the methods described herein, the antibody or fragment can be administered in a single daily dose on at least one day of the dosing regimen. The antibody or fragment can be administered in a single daily dose on each day of the dosing regimen. The antibody or fragment can be administered more than once a day on at least one day of the dosing regimen. The antibody or fragment can be administered more than once a day on each day of the dosing regimen. The interval between administrations can be at least one hour. The antibody or fragment can be administered over a period of time on at least one day of the dosing regimen.

In any of the methods described herein, the antibody or fragment can administered with a pharmaceutically acceptable carrier or diluent.

In any of the methods described herein, the antibody or fragment can be administered in conjunction with a therapeutic agent.

In any of the methods described herein relating to diabetes, the treatment can results in an increase in the average daily dose of insulin of no more than 10% of the patient's pre-dose amount of insulin six months after the treatment; an HbA1c of less than 7.5% one year after the treatment; or a C-peptide response to a mixed-meal tolerance test (MMTT) twelve months after the treatment that is at least 90% of the C-peptide response to MMTT in said patient before the treatment.

In any of the methods described herein relating to diabetes, the patient can be re-dosed if the average daily dose of insulin has increased by 50% or more; autoantibodies against one or more islet cell antigens are detected; islet cell antigen specific T cells are detected; beta-cell mass decreases by 50% or more; or the incidence of hypoglycemic or ketoacidosis episodes increases by 1 or more incidents per day in the patient at least 2 years after initial administration of the course of treatment.

In the methods described herein relating to re-treating, the at least one course of treatment can include a dosing regimen of an anti-CD3 antibody or antigen-binding fragment thereof, wherein the anti-CD3 antibody or fragment does not bind or has reduced binding to at least one Fc (gamma) receptor compared to the IgG1 immunoglobulin molecule produced by the cell line ARH-77 deposited under ATCC catalog number CRL-1621, and wherein over said course of treatment, the total amount of the anti-CD3 antibody or fragment thereof administered to said patient does not exceed 300 μg/kg when administered intravenously, and when administered other than intravenously, the total amount administered does not exceed the bioequivalent of intravenous administration of 300 μg/kg.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a boxplot of ADRR by tertile of time-normalized stimulated C-peptide AUC (nmol/L/min) Boxes represent the 25th-75th percentile. Circles represent values that are >1.5 times the interquartile range.

FIG. 2 is a boxplot of MAGE by tertile of time-normalized stimulated C-peptide AUC (nmol/L/min) Boxes represent the 25th-75th percentile. Circles represent values that are >1.5 times the interquartile range.

FIGS. 3A and 3B are graphs of glucose levels (mg/dL) from representative subjects over 29 days. In FIG. 3A, glucose levels are shown from subjects representing tertile 1 (ADRR=19.3; C-peptide AUC=0.43 nmol/L/min) In FIG. 3B, glucose levels are shown from subjects representing tertile 3 (ADRR=10.0; C-peptide AUC=2.58 nmol/L/min).

FIGS. 4A, 4B, and 4C are scatter plots of IDAA1C versus C-peptide AUC (4A), daily insulin dose versus C-peptide AUC (4B), and HbA1c versus C-peptide AUC (4C).

FIG. 5 is a box plot comparing the C-peptide AUC in subjects with an IDAA1c score of <9 to subjects with an IDAA1c of >9.

### DETAILED DESCRIPTION

In general, this document provides methods for treating immune-related diseases in mammals (e.g., human patients) using immunotherapy, methods of selecting patients for which immunotherapy is a suitable method of treatment, as well as diagnostic tests and assays for identifying biomarkers of immune-related disease and/or pathogenic immunological activity. As used herein, the term “immunotherapy” refers to therapy that results in elimination of, or a decrease in, a pathogenic effector cell activity (e.g., either by a modulation in activity or number directly in the cell or indirectly via alterations in products or cytokines that cells may secrete or be influenced by) in a subject. The effector cell can be a T cell (e.g., a CD4+ or a CD8+ T cell), a B cell, or an NK cell. The therapy can act by decreasing the activity per se of an effector cell (e.g., a cytotoxic T lymphocyte (CTL), a Th1 helper cell, a Th2 helper, a Th0 helper cell, or an antibody-producing plasma cell) or by increasing the activity per se of a suppressive cell (e.g., a Treg cell or a suppressor T cell). Alternatively, the therapy can act by decreasing an immune response (e.g., a CD4 and/or CD8 T cell and/or a B cell and/or a NK cell response) in which such an effector cell is generated or increasing an immune response in which such a suppressive cell is generated.

### Methods of Treating Immune-Related Diseases

Provided herein are methods of treating patients having, or suspected of having, immune-related diseases. As used herein, treating refers to reducing the severity of the disease or slowing progression of the disease. The term “immune-related disease” is used herein to refer to a disease that is associated with at least one abnormal immunogical activity. In some embodiments, an immune-related disease is an autoimmune disease. An autoimmune disease typically results when the subject's immune system is activated against one or more components (cells, tissues, or cell/tissue-free molecules) of the subject and attacks that subject's own organs, tissues or cells, instead of attacking, for example, foreign bacteria, viruses, and other infectious agents or cancer cells. Every mammalian subject exhibits autoimmunity to some extent, but such autoimmunity normally does not result in a disease state since the immune system regulates and suppresses normal autoimmunity. Autoimmune diseases develop when there is a disruption in the immune system's regulation. Autoimmune diseases can also result when there is a molecular alteration in a subject's cell that is recognized by the immune system, such that the immune system recognizes the altered cell as “foreign.”

Exemplary immune-related diseases include, but are not limited to, acute disseminated encephalomyelitis (ADEM), acute necrotizing hemorrhagic leukoencephalitis, Addison's disease, Agammaglobulinemia, allergic asthma, allergic rhinitis, alopecia greata, amyloidosis, ankylosing spondylitis, antiphospholipid syndrome, anti-GBM/anti-TBM nephritis, autoimmune diseases of the adrenal gland, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune neutropenia, autoimmune pancreatitis, autoimmune retinopathy, autoimmune oophoritis and orchitis, autoimmune thrombocytopenia, autoimmune thyroid disease, axonal & neuronal neuropathies, Balo disease, Behcet's disease, bullous pemphigoid, cardiomyopathy, Castleman disease, celiac sprue-dermatitis, Chagas disease, chronic fatigue immune dysfunction syndrome (CFIDS), chronic inflammatory demyelinating polyneuropathy, chronic recurrent multifocal ostomyelitis (CRMO), Churg-Strauss syndrome, cicatrical pemphigoid, CREST syndrome, cold agglutinin disease, Crohn's disease, Cogans syndrome, congenital heart block, coxsackie myocarditis, demyelinating neuropathies, dermatitis herpetiformis, dermatomyositis, Devic's disease (neuromyelitis optica), diabetes (e.g., type 1 diabetes, type 2 diabetes, or Latent Autoimmune Diabetes in Adults (LADA) (also referred to as late-onset type 1 diabetes, adult-onset type 1 diabetes, type 1.5 diabetes, slowly progressive insulin dependent diabetes mellitus, latent type 1 diabetes, youth-onset diabetes of maturity, latent-onset type 1 diabetes, and antibody-positive non-insulin-dependent diabetes)), discoid lupus, Dressler's syndrome, endometriosis, eosinophilic fasciitis, erythema nodosum, experimental allergic encephalomyelitis, Evans syndrome, essential mixed cryoglobulinemia, fibromyalgia-fibromyositis, fibrosing alveolitis, glomerulonephritis, Goodpasture's syndrome, Graves' disease, Graves' ophthalmopathy, Guillain-Barre, Hashimoto's encephalitis, Hashimoto's thyroiditis, hemolytic anemia, Henoch-Schonlein purpura, herpes gestationis, hypogammaglobulinemia, IgG4-related sclerosing disease, immunoregulatory lipoproteins, inclusion body myositis, idiopathic pulmonary fibrosis, idiopathic thrombocytopenia purpura (ITP), irritable bowel disease (IBD), IgA nephropathy, IgA neuropathy, interstitial cystitis, juvenile arthritis, Kawasaki syndrome, Lambert-Eaton syndrome, leukocytoclastic vasculitis, lichen planus, lichen sclerosus, ligneous conjunctivitis, linear IgA disease (LAD) lupus erythematosus, Lyme disease (chronic), Meniere's disease, microscopic polyangiitis, mixed connective tissue disease, Mooren's ulcer, Mucha-Habermann disease, multiple sclerosis, myasthenia gravis, myositis, narcolepsy, neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus vulgaris, peripheral neuropathy, perivenous encephalomyelitis, pernicious anemia, POEMS syndrome, polyarteritis nodosa, polychrondritis, polyglandular syndromes, polymyalgia rheumatics, polymyositis and dermatomyositis, primary agammaglobulinemia, primary biliary cirrhosis, postmyocardial infarction syndrome, postpericardiotomy syndrome, progesterone dermatitis, primary sclerosing cholangitis, psoriasis, psoriatic arthritis, idiopathic pulmonary fibrosis, pyoderma gangrenosum, pure red cell aplasia, Raynauld's phenomenon, Reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff-man syndrome, subacute bacterial endocarditis (SBE), Susac's syndrome, sympathetic ophthalmia, systemic lupus erythematosus, takayasu arteritis, temporal arteritis/giant cell arteritis, thrombotic thrombocytopenic purpura (TTP), Tolosa-Hunt syndrome, transverse myelitis, ulcerative colitis, undifferentiated connective tissue disease (UCTD), uveitis, vasculitides such as dermatitis herpetiformis vasculitis, vesiculobullous dermatosis, vitiligo, and Wegener's granulomatosis.

A subject “suspected of having an immune-related disease” is one having one or more signs of the disease. Signs of such diseases are well-known to those of skill in the art and include, without limitation, redness, swelling (e.g., swollen joints), skin rashes, joint pain, joint pain, loss of joint function, fever, chills, fatigue, loss of energy, headaches, loss of appetite, muscle stiffness, insomnia, itchiness, stuffy nose, sneezing, coughing, or one or more neurologic symptoms such as weakness, paresthesias, paralysis, dizziness, seizures, or pain. Signs of diabetes include, without limitation, higher than normal frequency of urination, unusual thirst, extreme hunger, unusual weight loss, extreme fatigue, visual problems, and irritability. In addition to these signs of diabetes (e.g., type 1 and, type 2 diabetes), subjects with type 1 and type 2 diabetes can have frequent infections (e.g., recurring skin, gum, lung, ear, or bladder infections), blurred vision, cuts and/or bruises that are slow to heal, tingling and/or numbness in the hands and/or feet. In addition, a subject with a history of pre-diabetes is considered to be “suspected of having diabetes.” Thus, it is understood that while none of the above disease signs amount to markers of the relevant diseases, subjects “suspected of having an immune-related disease” are not all the subjects within a species of interest.

The methods described herein include treating a patient with immunotherapy if a marker (also referred to as a biomarker herein) of the immune-related disease (e.g., diabetes, psoriasis, rheumatoid arthritis, lupus, inflammatory bowel disease, ulcerative colitis, Crohn's disease, Graves' disease, or multiple sclerosis) and a marker of pathogenic immunological (e.g., T cell) activity can be identified in the patient. Identifying refers to either a qualitative (i.e., present or absent), semi-quantitative, or a quantitative assessment of a marker. Thus, in some embodiments, the marker is identified (e.g., in a biological sample) and measured as a discrete value. Alternatively, it can be assessed and expressed using any of a variety of semi-quantitative/qualitative systems known in the art. For example, the marker can be identified as being, for example, (a) one or more of “excellent”, “good”, “satisfactory”, “unsatisfactory”, and/or “poor”; (b) one or more of “very high”, “high”, “average”, “low”, and/or “very low”; or (c) one or more of “++++”, “+++”, “++”, “+”, “+/−”, and/or “−”. It is understood that any particular marker can be a marker of an immune-related disease and a marker of pathogenic immunological activity.

For example, in one embodiment, the immune-related disease is rheumatoid arthritis. Biomarkers of rheumatoid arthritis can include one or more of the following: follistatin-like-protein-1, C-reactive protein (CRP), erythrocyte sedimentation rate (ESR), serum amyloid A, IL-6, S10B0, osteopontin, MMP-1, MMP-3, hyaluronic acid, and a product of collagen metabolism. Follistatin-like-protein 1 can be identified in the serum and synovial fluid of systemic-onset juvenile rheumatoid arthritis patients. See Wilson et al., Arthritis Rheum. 2010 Mar 30. [Epub ahead of print].

CRP is a protein that is found in blood and is a marker of acute inflammation. CRP can be identified by a variety of tests, including, for example, ELISA, immunoturbidimetry, rapid immunodiffusion, and visual agglutination. CRP levels are elevated (>1 mg/dL, typically >10 mg/dL) in patients with rheumatoid arthritis. The normal plasma concentration of CRP is <3 μg/ml in 90% of the healthy population, and <10 μg/ml in 99% of healthy individuals.

ESR (also referred to as a sedimentation or sedrate) is another test for inflammation. An increased ESR indicates non-specific inflammation in the body.

Serum amyloid A (SAA) is a protein that is predominantly synthesized by the liver during the acute phase of inflammation. SAA can be detected by a variety of tests, including a competitive type or a sandwich type immunoassay. See, for example, U.S. Patent Publication No. 20070298518.

Interleukin-6 (IL-6) is a 21 kDa secreted protein that has numerous biological activities, including activities involved in hematopoiesis and activation of the innate immune response. IL-6 is an acute-phase reactant and stimulates the synthesis of a variety of proteins, including adhesion molecules. Its major function is to mediate the acute phase production of hepatic proteins, and its synthesis is induced, e.g., by the cytokines IL-1 and TNF-α. IL-6 is normally produced by macrophages and T lymphocytes. The normal serum concentration of IL-6 is <5 pg/ml. Increased serum levels of IL-6 are indicative of inflammation.

Osteopontin (OPN) is a secreted, highly acidic, calcium-binding, phosphorylated glycoprotein. There are three isoforms that originate from alternative splicing, and are either free or bound to the extracellular matrix. See Take et al., Arthritis Rheum. 60(12):3591-601 (2009). Osteopontin expression increases in rheumatoid arthritis patients.

The matrix-metalloproteinase (MMP) family degrades almost all components of the extracellular matrix. Elevated levels of MMPs have been related to inflammatory processes in rheumatoid arthritis. MMP-1 and MMP-3 are produced by fibroblasts, osteoblasts, and endothelial cells upon stimulation by pro-inflammatory cytokines such as IL-1 or TNF-α. MMP-1 and MMP-3 have been detected in synovial fluid of RA-patients and the levels are responsive to anti-TNF-α therapy.

S100-proteins are members of a family of Ca²⁺-binding proteins that includes at least 20 members. The physiologically relevant structure of S100-proteins is a homodimer but some S100-proteins also can form heterodimers with each other, e.g. S100A8 and S100A9. S100A8, S100A9, the heterodimer S100A8/A9, and S100A12 have been found in inflammation. S100A8 is increased in chronic inflammation, while S100A9, S100A8/A9, and S100A12 are increased in acute inflammation. S100A8, S100A9, S100A8/A9, and S100A12 have been linked to different diseases with inflammatory components including rheumatoid arthritis. See, e.g., Burmeister and Gallacchi, Inflammopharmacology 3:221-230 (1995); and Foell et al., Rheumatology 42:1383-1389 (2003).

The glycosaminoglycan hyaluronic acid is one of the macromolecules essential for the function of a joint. It is synthesized by fibroblasts and other specialized connective tissue cells, and is one of the main components of the extracellular matrix. High concentrations of hyaluronic acid are found in synovial fluid where it is responsible for the retention of water thereby contributing to the lubrication of joints. In rheumatoid arthritis, the synthesis of hyaluronic acid is stimulated by the proinflammatory mediators IL-1 and TNF-α leading to increased body fluid (e.g., serum/plasma) levels. See, Sawai and Uzuki, Connective Tissue 33 (2001) 253-259).

Products of collagen metabolism include pyridinoline (PYD), which stabilizes collagen by cross-linking the strands of the collagen triple helix. The chemical structure of PYD is very stable and can be found in serum, urine, synovial fluid, and synovial tissue as an end product of collagen degradation. See, for example, Knott and Bailey, Bone 22: 181-187 (1998)). Elevated levels of PYD (e.g., in urine) have been linked to arthritis (see Kaufmann et al., Rheumatology 42 (2003) 314-320). PYD monitors cartilage involvement of joint destruction since it is released from cartilage and only to some degree from bone. See, for example, U.S. Patent Publication No. 20070298518.

Markers of pathogenic immunological cell activity in rheumatoid arthritis patients can include the presence of anti-cyclic citrullinated protein (CCP) autoantibodies and/or rheumatoid factors (RF). Anti-CCP antibodies are autoantibodies that can be detected in the serum of rheumatoid arthritis patients. Anti-CCP antibodies can be detected with a variety of tests, including a commercially available ELISA from INOVA Diagnostics Inc (San Diego, Calif.) in which values above 25 U/ml are considered positive for anti-CCP. See also Schellekens et al., Arthritis Rheum. 43 (2000) 155-163; and U.S. Patent Publication No. 20070298518.

RF are autoantibodies directed against the Fc portion of IgG and are found in the serum of approximately 80% of patients with rheumatoid arthritis and approximately 70% of patients with Sjögren's syndrome. RF can be detected by a variety of tests, including hemagglutination, latex agglutination, nephelometry, turbidimetry, or ELISA, including commercially available ELISA tests from IMMCO Diagnostics (Buffalo, N.Y.) and Sigma Chemical Company (St. Louis, Mo.). See, for example, Bas et al., Ann Rheum Dis 61:505-510 (2002).

Non-limiting examples of markers of pathogenic immunological (e.g., T cell) activity include the presence of: autoantigen-responsive T cells in lymphocytes from the subject (e.g., from a lymph node or peripheral blood); abnormal levels of one or more cytokines (e.g., interleukin-6 (IL-6), tumor necrosis factor-α (TNFα), interferon-γ (IFNγ), or interleukin-10 (IL-10)) in a body fluid (e.g., serum or plasma)); or autoreactive T cells expressing particular autoantigen-specific T cell receptors (TCR). Assays for autoantigen responsive T cells include in vitro proliferation assays and in vitro functional assays such as cytotoxic T lymphocyte (CTL) assays and assays to test for cytokine production. The proliferation, CTL, and cytokine producing assays are performed in the presence of an autoantigen of interest or one or more peptide epitopes derived from the autoantigen. T cells expressing TCR specific for an autoantigen can be tested, for example, by fluorescence flow cytometry using multimers (e.g., tetramers) of a peptide-MHC (major histocompatibility complex) molecule complex in which the peptide is derived from an autoantigen of interest using methods known in the art.

Thus, in the methods described herein, a patient with rheumatoid arthritis can be treated with immunotherapy when one or more markers of rheumatoid arthritis (e.g., follistatin-like-protein-1, CRP, ESR, SAA, IL-6, 5100, osteopontin, MMP-1, MMP-3, hyaluronic acid, or a product of collagen metabolism) and one or more markers of pathogenic immunological activity (e.g., anti-CCP antibodies or RF, or other pathological immunological activity described above) are identified in the patient. Suitable immunotherapies are described below.

In one embodiment, the immune-related disease is Graves' disease. A biomarker of Graves' disease includes increased levels of soluble cytotoxic T-lymphocyte associated 4 (CTLA-4) in the serum. See, Daroszewski et al., Eur J Endocrinol. 161(5):787-93 (2009). Another biomarker of Graves' disease includes the presence of TRAB-stimulating antibodies to the thyroid-stimulating hormone (TSH) receptor in the serum. Thus, in the methods described herein, a patient with Graves' disease can be treated with immunotherapy when a marker of Grave's disease such as soluble CTLA-4 or TRAB-stimulating antibodies and one or more markers of pathogenic immunological activity are identified in the patient. Suitable immunotherapies are described below.

In one embodiment, the immune-related disease is psoriasis. Biomarkers of psoriasis include platelet P-selectin or soluble P-selectin. Platelet P-selectin can be assessed by flow-cytometry while soluble P-selectin can be measured by an ELISA. See, Garbaraviciene et al., Exp Dermatol. 2010 May 13 [Epub ahead of print]. Thus, in the methods described herein, a patient with psoriasis can be treated with immunotherapy when a marker of psoriasis such as soluble P-selectin and one or more markers of pathogenic immunological activity are identified in the patient. Suitable immunotherapies are described below.

In one embodiment, the immune-related disease is diabetes. Biomarkers of diabetes include, for example, amylin, glucagon, islet-associated protein, and insulin production. Amylin is a neurohormone that is co-secreted with insulin from the beta cells of the pancreas, and its concentrations are abnormally low in patients with diabetes. See, Koda et al., Lancet, 339(8802):1179-80 (1992). Amylin can be detected using a radioimmunoassay such as with the kit from Phoenix Pharmaceuticals Inc. (Belmont, Calif.).

Glucagon is a hormone involved in glucose homeostasis and normally limits the severity of hypoglycemic events. In diabetic patients, the normal increase of glucagon with hypoglycemia is lost and the normal decrease of glucagons with hyperglycemia is lost. See, Brown et al., Diabetes Care, 31:1403-1404 (2008).

Islet-associated proteins in the glutamic acid decarboxylase (GAD) pathway, such as GAD65, and insulin granule membrane proteins, including those in the protein tyrosine phosphatase pathway (e.g., IA-2 and IA-2B), and antibodies thereto signal inflammation of the islet cells and β-cell killing and are markers associated with diabetes.

Insulin production is another marker of diabetes. Typically, insulin production is monitored by detecting C-peptide, which is produced by cleavage of proinsulin and accordingly, produced at the same rate as insulin.

Additional markers of diabetes include glucose tolerance, glucose variability, HbA1c (glycosylated hemoglobin), and pancreatic islet inflammation. For example, patients with diabetes can have an abnormal glucose tolerance test. In healthy controls, fasting plasma glucose (measured before an oral glucose tolerance test (OGTT)) is <6.1 mmol/l (110 mg/dl). Patients having fasting levels between 6.1 and 7.0 mmol/l (110 and 125 mg/dl) are considered to have impaired fasting glycemia while fasting levels repeatedly at or above 7.0 mmol/l (126 mg/dl) are diagnostic of diabetes. With a 2 hour OGTT test, glucose levels are below 7.8 mmol/l (140 mg/dl) in healthy controls. Levels between 7.8 (140 mg/dL) and 11.1 mmol/l (200 mg/dl) indicate impaired glucose tolerance, while glucose levels above 11.1 mmol/l (200 mg/dl) at 2 hours confirms a diagnosis of diabetes.

Increased glucose variability also is a marker of diabetes. Glucose variability refers to the variability in glucose concentration over time. Glucose variability can be expressed as the average daily risk range (ADRR) or mean amplitude of glycemic excursions (MAGE). See, Kovatchev et al., Diabetes 29:2433-38 (2006); and Service et al., Diabetes, 19:644-655 (1970). As shown in the Example herein, increased glucose variability is associated with increased risk of long and short-term complications of diabetes.

HbA1c also is a marker of diabetes and can be used to identify the average plasma glucose concentration over prolonged periods of time. HbA1c can be measured by a variety of tests, including immunoassays and high performance liquid chromatography. Healthy controls have an HbA1c content of about 4%-5.9%, while pre-diabetic and diabetic patients have higher levels. Insulin dose-adjusted HbA1c also can be measured as described in Example 2.

Pancreatic islet inflammation can be detected using magnetic resonance imaging with ferromagnetic particles. See, for example, Turvey et al., J Clin Invest. 115(9):2454-61 (2005), Epub 2005 Aug 18.

Markers of pathogenic immunological activity in diabetes patients can include the presence of anti-glutamatic acid decarboxylase autoantibodies, protein tyrosine phosphatase-like protein autoantibodies (anti-IA-2), zinc transporter (ZNT8) autoantibodies, or insulin autoantibodies (IAA). Anti-ZNT8 (islet beta-cell secretory granule membrane protein) autoantibodies have been found in patients with adult-onset autoimmune diabetes. See, Lampasona et al., Diabetes Care. 33(1):104-8 (2010). Autoantibodies can be detected using a variety of techniques, including, for example, ELISA, radioimmunoprecipitation, or radioimmunoassays.

Thus, in the methods described herein, a patient with diabetes can be treated with immunotherapy when one or more marker of diabetes such as amylin, glucagon, islet-associated protein, insulin production, glucose tolerance, glucose variability, HbA1c, and pancreatic islet inflammation, and one or more markers of pathogenic immunological activity (e.g., autoantibodies, abnormal levels of one or more cytokines, or autoantigen reactive T lymphocytes) are identified in the patient. Suitable immunotherapies are described below.

Methods described herein can include monitoring the patient to, for example, determine if the immune-related disease is improving with immunotherapy. A patient may need to be re-dosed with one or more additional courses of immunotherapy (e.g., with an anti-CD3 antibody or antigen-binding fragment thereof using any of the dosing regimens described herein) if an indicator of return to active disease is identified. Any method can be used to monitor indicators of return to active disease in the patient. For example, for diabetes patients, indicators of return to active disease can include increased insulin requirements; a change of HbA1c; a change in insulin dose-adjusted HbA1c; a change in C-peptide measurements (e.g., fasting C-peptide); a change in glucose tolerance tests; a change in glucose variability; increased incidence of abnormal glucose measurements; detection of autoantibodies against one or more islet cell antigens; detection of islet cell antigen specific T cells; a decrease in beta-cell mass; or increased incidence of hypoglycemic or ketoacidosis episodes. In one embodiment, a patient is re-dosed if the average daily dose of insulin increases by 50% or more, if autoantibodies against one or more islet cell antigens, if islet cell antigen specific T cells are detected, if beta cell mass decreases (e.g., by 50% or more), or if the incidence of hypoglycemic or ketoacidosis episodes increases by one or more incidents per day in the patient at least two years after initial administration of the course of treatment. In one embodiment, treatment for diabetes can result, for example, in an increase in the average daily dose of insulin of no more than 10% of the patient's predose amount six months after treatment, HbA1c is less than 7.5% one year after treatment, or a C-peptide response to a mixed meal tolerance test (MMTT) twelve months after the treatment that is at least 90% of the C-peptide response to MMTT in the patient before the treatment.

For RA patients, joint pain and/or stiffness, bone erosion, ESR, and/or CRP can be monitored in the patient. For multiple sclerosis patients, lower extremity function, upper extremity function, vision, and cognitive function can be monitored. Magnetic resonance imaging (e.g., fluid-attenuated inversion recovery) can be performed to examine lesions and to differentiate old lesions from new or active lesions. Evoked potential tests can be performed to monitor nerve transmission.

### Immunotherapy

Once a patient is identified as a candidate for therapy, an effective course of immunotherapy can be administered to the patient. For example, an antibody, or antigen binding fragment thereof, that binds to an immunomodulatory molecule on the surface of an immune cell can be administered to the patient. As used herein, an “immunomodulatory molecule” is a molecule involved in the transduction of a signal to the metabolic machinery of a cell. The signal can be an activity-enhancing signal, an activity-suppressing signal, or inactivating (e.g., anergizing) signal. Thus, immunomodulatory molecules include cytokines (e.g., interleukin-2 (IL-2), TNFα, IL-6, and IFN-γ), receptors (e.g., 4-1BB, CD28, and GITR) or ligands (4-1BB ligand, B-7.1, and B-7.2) on the surface of a cell, or accessory molecules that do not necessarily interact with a receptor or a ligand (e.g., CD3 molecules). From this description it will be clear that immunomodulatory molecules include cytokines and their receptors, costimulatory molecules and their receptors, and signal transducing molecules that do not interact with a receptor or a ligand. The immunomodulatory molecule-binding agents (e.g., antibodies, antigen-binding fragments of antibodies, soluble receptors or ligands or functional fragments of receptors or ligands) used in the methods of the present document can be agonists or antagonists.

As used herein, an “immune cell” is a cell and all varieties of their subsets based on surface markers or unique function, involved at any level of an immune response. Thus, major classes of immune cells include CD4+ and CD8+ T lymphocytes (with any of a variety of functions), B lymphocytes, NK cells, cells of the macrophage/monocyte lineage including macrophages, monocytes, dendritic cells, and Langerhans cells, and granulocytic cells (e.g., basophils, eosinophils, and polymorphonuclear (PMN) cells).

“Antibody” as the term is used herein refers to a protein that generally comprises heavy chain polypeptides and light chain polypeptides. IgG, IgD, and IgE antibodies comprise two heavy chain polypeptides and two light chain polypeptides. IgA antibodies comprise two or four of each chain and IgM generally comprise 10 of each chain. Single domain antibodies having one heavy chain and one light chain and heavy chain antibodies devoid of light chains are also contemplated. A given antibody comprises one of five types of heavy chains, called alpha, delta, epsilon, gamma and mu, the categorization of which is based on the amino acid sequence of the heavy chain constant region. These different types of heavy chains give rise to five classes of antibodies, IgA (including IgA1 and IgA2), IgD, IgE, IgG (IgG1, IgG2, IgG3 and IgG4) and IgM, respectively. A given antibody also comprises one of two types of light chains, called kappa or lambda, the categorization of which is based on the amino acid sequence of the light chain constant domains.

“Antigen binding fragment” and “antigen binding antibody fragment” as the terms are used herein refer to an antigen binding molecule that is not an antibody as defined above, but that still retains at least one antigen binding site. Thus, in one embodiment, an antigen binding fragment or antigen binding antibody fragment of an anti-CD3 antibody is a fragment or antibody fragment that binds to CD3. Antibody fragments often comprise a cleaved portion of a whole antibody, although the term is not limited to such cleaved fragments. Antigen binding antibody fragments can include, for example, Fab fragments, F(ab′)2 fragments, scFv fragments, diabodies, linear antibodies, multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelid antibodies, camelized antibodies, and V_{HH} containing antibodies.

“Humanized antibody” as the term is used herein refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those of ordinary skill in the art will be aware of humanized antibodies, and will also be aware of suitable techniques for their generation.

“Chimeric antibody” as the term is used herein refers to an antibody that has been engineered to comprise a human constant region. Chimeric antibodies are typically less immunogenic to humans, relative to non-chimeric antibodies, and thus offer therapeutic benefits in certain situations. Those of ordinary skill in the art will be aware of chimeric antibodies, and will also be aware of suitable techniques for their generation.

In some embodiments, an agent is administered to the patient that includes a soluble ligand or receptor, or a functional fragment thereof, that binds to an immunomodulatory molecule on the surface of an immune cell. As used herein, a “functional fragment of a receptor” is a fragment of the receptor that is shorter than the naturally occurring, full-length, mature receptor polypeptide but has at least 25% (e.g., at least: 30%; 40%; 50%; 60%; 70%; 80%; 90%; 95%; 98%; 99%; or even 100% or more) of the ability of the naturally occurring, full-length, mature receptor polypeptide to bind to its natural ligand.

As used herein, a “functional fragment of a ligand” is a fragment of the ligand that is shorter than the naturally occurring, full-length, mature ligand polypeptide but has at least 25% (e.g., at least: 30%; 40%; 50%; 60%; 70%; 80%; 90%; 95%; 98%; 99%; or even 100% or more) of the ability of the naturally occurring, full-length, mature ligand polypeptide to bind to its natural receptor.

In one embodiment, the immunomodulatory molecule is a molecule of the CD3 complex, a T cell receptor molecule, a CD4 molecule, a CD8 molecule, a CD20 molecule, a B7 molecule, or a 4-1BB molecule. For example, immunotherapy can include treatment with an anti-CD3 antibody or antigen-binding fragment thereof. Any of a variety of anti-CD3 antibodies or fragments thereof can be employed in the dosing regimens described herein for treating patients with immune-related disease. In certain embodiments, the antibody or antigen binding fragment thereof is a human antibody or fragment. In certain embodiments, the antibody or antigen binding fragment thereof is a non-human antibody or fragment, e.g., a mouse or rat antibody or fragment. In certain embodiments, the antibody or fragment thereof is chimeric in that it contains human heavy and/or light chain constant regions. In certain embodiments, the antibody or fragment thereof is humanized in that it contains one or more human framework regions in the variable region together with non-human (e.g., mouse, rat, or hamster) complementarity-determining regions (CDRs) of the heavy and/or light chain. In certain embodiments, the antibody is monoclonal. In certain embodiments, the fragment is derived from a monoclonal antibody (e.g., cleaved at its hinge region to generate a F(ab′)₂ fragment). In certain embodiments, the antibody is a polyclonal antibody population in that it comprises a plurality of different antibodies, each of which binds to the same antigen, many with different affinities and may bind to different epitopes on the same target antigen. In certain embodiments, the fragment is derived from a polyclonal antibody population.

In certain embodiments, an antibody antigen-binding fragment is a Fab fragment, a F(ab′)₂ fragment, a scFv fragment, a diabody, a linear antibody, a multispecific antibody fragment such as a bispecific, a trispecific, or a multispecific antibody (e.g., a diabody, a triabody, a tetrabody), a minibody, a chelating recombinant antibody, a tribody or bibody, an intrabody, a nanobody, a small modular immunopharmaceutical (SMIP), a binding-domain immunoglobulin fusion protein, a camelid antibody, or a V_{HH} containing antibody.

In certain embodiments, an anti-CD3 antibody or antigen-binding fragment thereof to be employed in one or more of the dosing regimens disclosed herein binds a human CD3 polypeptide. A variety of anti-human CD3 antibodies and fragments are known in the art. Such antibodies and fragments are useful, for example, when the animal to be treated is a human. In certain embodiments, an antibody or fragment thereof to be employed in one or more of the dosing regimens disclosed herein binds a non-human CD3. For example, a non-human mammal may be administered an anti-CD3 antibody or fragment, which antibody or fragment thereof binds a CD3 polypeptide present in that animal Any of a variety of non-human mammals are known, and can be administered an anti-CD3 antibody or fragment thereof that binds a CD3 present in such that animal. Non-limiting examples include dogs, cats, cows, horses, sheep, goats, pigs, mice, rats, non-human primates, and hamsters. The anti-CD3 antibodies can of the same species or different species. Moreover, they can be analogous to the chimeric and humanized antibodies described herein. Thus, when treating a horse, for example, the CD3 antibody can contain heavy and/or light chain variable regions of another species (e.g., mouse, rat, hamster, or human) and horse heavy and/or light chain constant regions (chimeric heavy and/or light chains). Alternatively, heavy and/or light chains can contain all the CDRs from another species (as above) with the rest of the heavy and/or light chain being horse (horse analogs of humanized heavy and light chains). Moreover, the heavy chain or the light chain can of the chimeric type and the other chain can be of the horse analog of the humanized chain. The same principles apply to anti-CD3 antibodies for use in any of the exemplary species listed above.

In certain embodiments, an anti-CD3 antibody or antigen-binding fragment thereof to be employed in one or more of the dosing regimens disclosed herein binds a CD3 epsilon polypeptide, e.g., a human CD3 epsilon polypeptide. In certain embodiments, an anti-CD3 antibody or fragment thereof to be employed in one or more of the dosing regimens disclosed herein binds a CD3 gamma polypeptide, e.g., a human CD3 gamma polypeptide. In certain embodiments, an anti-CD3 antibody or fragment thereof to be employed in one or more of the dosing regimens disclosed herein binds a CD3 delta polypeptide, e.g., a human CD3 delta polypeptide. In certain embodiments, an anti-CD3 antibody or fragment thereof to be employed in one or more of the dosing regimens disclosed herein binds a CD3 zeta polypeptide, e.g., a human CD3 zeta polypeptide.

In certain embodiments, an antibody to be employed in one or more of the dosing regimens disclosed herein is otelixizumab, a humanized aglycosylated antibody. Otelixizumab, also known as TRX4, comprises a heavy chain having the sequence set forth in SEQ ID NO: 1 EVQLLESGGGLVQPGGSLRLSCAASGFTFSSFPMAWVRQAPGKGLEWVSTISTSGGR TYYRDSVKGRFTISRDNSKNTLYLQMNSLRAEDTAVYYCAKFRQYSGGFDYWGQG TLVTVSSASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKSCDKTHTC PPCPAPELLGGPSVFLFPPKPKDTLMISRTPEVTCVVVDVSHEDPEVKFNWYVDGVE VHNAKTKPREEQYASTYRVVSVLTVLHQDWLNGKEYKCKVSNKALPAPIEKTISKA KGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK], and a light chain having the sequence set forth in SEQ ID NO:2 [DIQLTQPNSVSTSLGSTVKLSCTLSSGNIENNYVHWYQLYEGRSPTTMIYDDDKRPD GVPDRFSGSIDRSSNSAFLTIHNVAIEDEAIYFCHSYVSSFNVFGGGTKLTVLRQPKAA PSVTLFPPSSEELQANKATLVCLISDFYPGAVTVAWKADSSPVKAGVETTTPSKQSNN KYAASSYLSLTPEQWKSHRSYSCQVTHEGSTVEKTVAPTECS]. In certain embodiments, an antibody or fragment thereof to be employed in one or more of the dosing regimens disclosed herein comprises the heavy chain variable region of otelixizumab, as set forth in SEQ ID NO:3 [EVQLLESGGGLVQPGGSLRLSCAASGFTFSSFPMAWVRQAPGKGLEWVSTISTSGG RTYYRDSVKGRFTISRDNSKNTLYLQMNSLRAEDTAVYYCAKFRQYSGGFDYWGQ GTLVTVSS]. In certain embodiments, an antibody or antigen-binding fragment thereof to be employed in one or more of the dosing regimens disclosed herein comprises the light chain variable region of otelixizumab, as set forth in SEQ ID NO: 4 [DIQLTQPNSVSTSLGSTVKLSCTLSSGNIENNYVHWYQLYEGRSPTTMIYDDDKRPD GVPDRFSGSIDRSSNSAFLTIHNVAIEDEAIYFCHSYVSSFNVFGGGTKLTVLR].

In certain embodiments, an antibody or antigen-binding fragment thereof to be employed in one or more of the dosing regimens disclosed herein comprises one or more complementarity determining regions (CDRs) of otelixizumab. For example, an antibody or fragment thereof may include one or more of the following: the otelixizumab heavy chain variable complementarity determining region 1 (VH CDR1) comprising the amino acid sequence as set forth in SEQ ID NO: 5 [SFPMA], the otelixizumab heavy chain variable complementarity determining region 2 (VH CDR2) comprising the amino acid sequence as set forth in SEQ ID NO: 6 [TISTSGGRTYYRDSVKG], the otelixizumab heavy chain variable complementarity determining region 3 (VH CDR3) comprising the amino acid sequence as set forth in SEQ ID NO: 7 [FRQYSGGFDY], the otelixizumab light chain variable complementarity determining region 1 (VL CDR1) comprising the amino acid sequence as set forth in SEQ ID NO: 8 [TLSSGNIENNYVH], the otelixizumab light chain variable complementarity determining region 2 (VL CDR2) comprising the amino acid sequence as set forth in SEQ ID NO: 9 [DDDKRPD], or the otelixizumab light chain variable complementarity determining region 3 (VL CDR3) comprising the amino acid sequence as set forth in SEQ ID NO: 10 HSYVSSFNV]. In certain embodiments, the antibody or fragment thereof comprises each of the complementarity determining regions comprising the amino acid sequences set forth in SEQ ID NOs: 5-10.

In certain embodiments, an antibody or antigen-binding fragment thereof to be employed in one or more of the dosing regimens disclosed herein exhibits reduced binding to at least one Fc (gamma) receptor. In certain embodiments, binding of the modified antibody or fragment thereof to at least one Fc (gamma) receptor is reduced as compared to the binding exhibited by the OKT3 antibody. OKT3 is a mouse antibody that is well-known to those of ordinary skill in the art. OKT3 binds the CD3 antigen, and is available from a variety of commercial sources (e.g., eBioscience™ at www.ebioscience.com). Additionally, a hybridoma cell line expressing the OKT3 antibody has been deposited under ATCC number CRL-8001. In certain embodiments an antibody or fragment thereof to be employed in one or more of the dosing regimens disclosed herein exhibits at least 25% reduced binding to at least one Fc (gamma) receptor as compared to the binding that would be observed with the OKT3 antibody. For example, the antibody or fragment thereof may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, binding of the modified antibody or antigen-binding fragment thereof to at least one Fc (gamma) receptor is reduced as compared to the binding exhibited by the huOKT3-gamma-1 and/or huOKT3-gamm⁻¹(A318) antibodies as described in Xu et al., Cellular Immunology, 200, 16-26 (2000). In certain embodiments an antibody or fragment thereof to be employed in one or more of the dosing regimens disclosed herein exhibits at least 25% reduced binding to at least one Fc (gamma) receptor as compared to the binding that would be observed with the huOKT3-gamma-1 and/or huOKT3-gamm⁻¹(A318) antibodies. For example, the antibody or fragment thereof may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, binding of the modified antibody or antigen-binding fragment thereof to at least one Fc (gamma) receptor is reduced as compared to the binding exhibited by the IgG1 immunoglobulin molecule produced by the ARH-77 cell line deposited under ATCC catalog number CRL-1621. In certain embodiments an antibody or fragment thereof to be employed in one or more of the dosing regimens disclosed herein exhibits at least 25% reduced binding to at least one Fc (gamma) receptor as compared to the binding that would be observed with the IgG1 antibody produced by the ARH-77 cell line deposited under ATCC catalog number CRL-1621. For example, the antibody or fragment thereof may exhibit at least 25%, 30%, 35%, 40%, 45%, 50%, 55%, 60%, 65%, 70%, 75%, 80%, 85%, 90%, 95%, 96%, 97%, 98%, 99% or more reduced binding.

In certain embodiments, an antibody or antigen-binding fragment thereof to be employed in one or more of the dosing regimens disclosed herein does not bind (e.g., exhibits no detectable binding) to at least one Fc (gamma) receptor.

In certain embodiments, an antibody or antigen-binding fragment thereof that exhibits reduced binding to at least one Fc (gamma) receptor comprises a modification that results in the reduced binding. In certain embodiments, such an antibody or fragment thereof may be modified at one or more amino acid residues within a heavy chain, a light chain, or both. The glycosylation state of an antibody or fragment thereof may affect its binding to one or more Fc (gamma) receptors. In certain embodiments, glycosylation of an antibody or fragment thereof is altered by modifying one or more amino acid residues within a heavy chain, a light chain, or both. For example, otelixizumab comprises a human IgG1 heavy chain constant region that has been modified by replacing an asparagine at position 297 of SEQ ID NO: 1 with an alanine. This modification results in loss of or decreased glycosylation of the antibody's Fc region and significantly decreased binding of the antibody to major Fc receptors, leading to decreased pro-inflammatory cytokine release and immunogenicity, and no perturbation of Epstein Barr Virus immunity. In certain embodiments, an antibody or fragment thereof comprises an alanine at an amino acid position corresponding to amino acid position 299 of SEQ ID NO: 1. Position 299 of SEQ ID NO: 1 corresponds to amino acid residue number 297 of IgG heavy chains, according to the Kabat numbering system (see Kabat E A, Wu T T, Perry H, Gottesman K, and Foeller C. (1991) Sequences of Proteins of Immunological Interest, Fifth Edition. NIH Publication No. 91-3242.) All IgG molecules contain a single conserved N-linked glycosylation site in each of their Cγ2 domains, which conserved glycosylation site corresponds to amino acid residue number 297 of IgG heavy chains, according to the Kabat numbering system (see Arnold et al., The Impact of Glycosylation on the Biological Function and Structure of Human Immunoglobulins, Annu. Rev. Immunol. 2007. 25:21-50, 2007). Thus, in certain embodiments, such an IgG conserved glycosylation site is modified so as to reduce or eliminate glycosylation.

Other amino acid modifications of anti-CD3 antibodies that result in reduced binding to at least one Fc (gamma) receptor are known in the art. For example, a humanized OTK3-derived antibody in which two amino acid residues at positions 234 and 235 of the Fc domain have been modified to alanine residues (referred to as hOKT3-gamma-1 (ala-ala)) is disclosed in U.S. Patent Publication Nos. 2007/0077246 and 2008/0095766. The hOKT3-gamma-1 (ala-ala) antibody is fully glycosylated but is described as exhibiting reduced binding to Fc (gamma) receptors.

Other examples of anti-CD3 antibodies include, without limitation, hOKT3 (humanized (IgG1 or IgG4) anti-human CD3), HUM291 (humanized (IgG2) anti-human CD3; visilizumab; NUVIONTM), UCHT1 (mouse (IgG1) anti-human CD3), Leu4 (mouse (IgG1) anti-human CD3), 500A2 (hamster (IgG) anti-mouse CD3), CLB-T3/3 (mouse (IgG2a) anti-human CD3), BMA030 (mouse (IgG2a) anti-human CD3), YTH 12.5 (rat (IgG2b) anti-human CD3), and NI-0401 (fully human anti-human CD3). Those of ordinary skill in the art will be aware of other anti-CD3 antibodies that can be used in accordance with the dosing regimens disclosed herein.

In certain embodiments, an antibody or antigen-binding fragment thereof that exhibits reduced binding to at least one Fc (gamma) receptor is modified in that it lacks some or all of an Fc domain. For example, Fab fragments and F(ab′)₂ fragments lack some or all of an Fc domain.

In certain embodiments, an antibody or antigen-binding fragment thereof is modified in some other way such that it exhibits reduced binding to at least one Fc (gamma) receptor. For example, the antibody or fragment thereof may be modified by covalent linkage of a chemical moiety that prevents the antibody or fragment thereof from binding at least one Fc (gamma) receptor. As another example, the antibody or fragment thereof may be modified by non-covalent linkage of a chemical moiety that prevents the antibody or fragment thereof from binding at least one Fc (gamma) receptor. Any of a variety of moieties may be covalently or non-covalently linked to the antibody or fragment thereof to prevent binding to at least one Fc (gamma) receptor. Those of ordinary skill in the art will be aware of suitable moieties that can be linked to an antibody or fragment, and will be able to employ such moieties in accordance with the teachings herein.

Those of ordinary skill in the art will be aware of other antibodies and antigen-binding fragments that exhibit reduced binding to at least one Fc (gamma) receptor, which antibodies and fragments can be employed in one or more of the dosing regimens disclosed herein.

“Dosing regimen,” as used herein, refers to the total course of treatment administered to an animal, e.g., treatment of a human with an anti-CD3 antibody or antigen-binding fragment thereof. In some embodiments, the total amount of the anti-CD3 antibody or fragment thereof administered to the patient does not exceed 300 μg/kg when administered intravenously, and when administered other than intravenously, the total amount administered does not exceed the bioequivalent of intravenous administration of 300 μg/kg. A dosing regimen may include a given number of days of treatment. For example, an anti-CD3 dosing regimen may include administering an anti-CD3 antibody to an animal for a minimum number of days, a maximum number of days, or a specific number of days. As non-limiting examples, an anti-CD3 antibody may be administered to an animal over a regimen of five days, eight days, fourteen days, or any number of days in between or beyond. An anti-CD3 dosing regimen may be as short as one day, although as will be apparent from the remainder of the present specification, multiple day dosing regimens permit administration of higher amounts of antibody on later days while significantly reducing cytokine release syndrome and other negative effects. Additionally and/or alternatively, a regimen may include a given amount of therapeutic agent administered per day. For example, an anti-CD3 antibody may be administered to an animal in a minimum amount on one or more days of the regimen, in a maximum amount on one or more days of the regimen, or in a specific amount on one or more days of the regimen. In certain embodiments, an anti-CD3 antibody or antigen-binding fragment can be administered as a continuous infusion (e.g., by a microinfusion pump or slow-release patch) rather than a fixed dose.

### Exemplary Dosing Regimens

In certain embodiments, a course of treatment with an anti-CD3 antibody or antigen-binding fragment thereof may be administered over a dosing regimen of one day, two days, three days, four days, five days, six days, seven days, eight days, nine days, ten days, eleven days, twelve days, thirteen days, fourteen days, or more. In certain embodiments, an anti-CD3 antibody or fragment is administered over a dosing regimen of five days. In certain embodiments, an anti-CD3 antibody or fragment is administered over a dosing regimen of eight days. In certain embodiments, an anti-CD3 antibody or antigen-binding fragment is administered as a continuous infusion (e.g., by a microinfusion pump or slow-release patch) rather than a fixed dose. Limiting the number of days of a dosing regimen can confer practical benefits on a patient being treated. For example, limiting a dosing regimen to five days may minimize the inconvenience to a patient when that patient needs to travel to a hospital or clinic to receive anti-CD3 antibody or fragment treatment. Limiting the number of days in a dosing regimen can also increase patient safety since fewer hospital visits will result in fewer medical recordkeeping requirements, and thus fewer chances of making recording or filing mistakes. Limiting the number of days in a given dosing regimen can also decrease the costs associated with treatment, since the treatment provider will need to spend less total time with the patient.

In certain embodiments, an anti-CD3 antibody or antigen-binding fragment thereof is administered on consecutive days during a given dosing regimen. In certain embodiments, an anti-CD3 antibody or fragment thereof is not administered on consecutive days of a dosing regimen. For example, a given dosing regimen may include one or more days in which an anti-CD3 antibody or fragment thereof is not administered. In certain embodiments, a dosing regimen comprises one, two, three, four, five, six, seven or more days in which an anti-CD3 antibody or fragment thereof is not administered. In certain embodiments, an anti-CD3 antibody or fragment thereof is administered every other day of a dosing regimen. In certain embodiments, an anti-CD3 antibody or fragment thereof is administered every third day, or every fourth day.

In certain embodiments, an anti-CD3 antibody or antigen-binding fragment thereof is administered in a low dose on at least one day of a dosing regimen. In certain embodiments, an anti-CD3 antibody or fragment thereof is administered in a low dose during the early portion of a dosing regimen, e.g., on the first one, two and/or three days of the regimen. As will be appreciated by those of ordinary skill in the art upon reading the present specification, administering the anti-CD3 antibody or fragment thereof in a low dose during the early portion of a dosing regimen facilitates the administration of higher individual doses later in a dosing regimen than would be possible with traditional dosing regimens. In certain embodiments, an anti-CD3 antibody or fragment thereof is administered in an amount that does not exceed about 0.5 mg per day during the early portion of a dosing regimen. For example, the anti-CD3 antibody or fragment thereof may be administered in an amount that does not exceed about 0.5 mg per day on the first one, two and/or three days of the regimen. In certain embodiments, the amount of anti-CD3 antibody or fragment thereof administered on the first two days of the dosing regimen does not exceed about 0.5 mg per day. In certain embodiments, the amount of anti-CD3 antibody or fragment thereof administered on the first day of the dosing regimen does not exceed about 0.5 mg. In certain embodiments, an anti-CD3 antibody or fragment thereof is administered in an amount that does not exceed about 0.45 mg per day, about 0.4 mg per day, about 0.35 mg per day, about 0.3 mg per day, about 0.25 mg per day, about 0.2 mg per day, about 0.15 mg per day, about 0.1 mg per day, about 0.09 mg per day, about 0.08 mg per day, about 0.07 mg per day, about 0.06 mg per day, about 0.05 mg per day, about 0.04 mg per day, about 0.03 mg per day, about 0.02 mg per day, about 0.01 mg per day, or less during the early portion of a dosing regimen, e.g. on the first one, two and/or three days of the regimen.

In certain embodiments, the amount of anti-CD3 antibody or antigen-binding fragment thereof administered on each of days one and two of a given dosing regimen does not exceed about 0.3 mg per day. In certain embodiments, the amount of anti-CD3 antibody or fragment thereof administered on each of days one and two of a given dosing regimen does not exceed about 0.2 mg per day. In certain embodiments, the amount of anti-CD3 antibody or fragment thereof administered on day one of a given dosing regimen is about 0.1 mg. In certain embodiments, the amount of anti-CD3 antibody or fragment thereof administered on day two of a given dosing regimen is about 0.2 mg. In certain embodiments, the amount of anti-CD3 antibody or fragment thereof administered on day two of a given dosing regimen is about 0.3 mg.

In certain embodiments, the amount of anti-CD3 antibody or antigen-binding fragment thereof administered increases between days two and five of a given dosing regimen. In certain embodiments, the amount of increase between days two and five is more than about 0.3 mg. For example, the amount of anti-CD3 antibody or fragment thereof administered may increase more than about 0.3 mg, more than about 0.35 mg, more than about 0.4 mg, more than about 0.45 mg, more than about 0.5 mg, more than about 0.55 mg, more than about 0.6 mg, more than about 0.65 mg, more than about 0.7 mg, more than about 0.75 mg, more than about 0.8 mg, more than about 0.85 mg, more than about 0.9 mg, more than about 0.95 mg, more than about 1.0 mg, more than about 1.1 mg, more than about 1.2 mg, more than about 1.3 mg, more than about 1.4 mg, more than about 1.5 mg, more than about 1.6 mg, more than about 1.7 mg, more than about 1.8 mg, more than about 1.9 mg, more than about 2 mg, more than about 2.5 mg, more than about 3 mg, more than about 3.5 mg, more than about 4 mg, more than about 4.5 mg, more than about 5 mg, or more.

In certain embodiments, the amount of anti-CD3 antibody or antigen-binding fragment thereof administered increases on each day between days two and five of a given dosing regimen such that the total increase between days two and five is more than about 0.3 mg. In certain embodiments, the amount of anti-CD3 antibody or fragment thereof administered between days two and five of a given dosing regimen increases by more than about 0.3 mg, but the amount of anti-CD3 antibody or fragment thereof administered does not increase on each day. For example, the amount of antibody or fragment thereof administered may remain constant or even decrease between, e.g., days two and three, days three and four, or days four and five, but the total amount nevertheless increases by more than about 0.3 mg between days two and five.

In certain embodiments, the amount of anti-CD3 antibody or antigen-binding fragment thereof administered on day three of a given dosing regimen is less than about 0.5 mg greater than the amount of anti-CD3 antibody or fragment thereof administered on day two of the dosing regimen. For example, the amount of antibody or fragment thereof administered on day three of the dosing regimen may be less than about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day two. In certain embodiments, the amount of antibody or fragment thereof administered on day three of the dosing regimen is about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day two. In certain embodiments, the amount of antibody or fragment thereof administered on day three of the dosing regimen is about equal to the amount administered on day two. In certain embodiments, the amount of antibody or fragment thereof administered on day three of the dosing regimen is less than the amount administered on day two. For example, the amount of antibody or fragment thereof administered on day three of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day two. In certain embodiments, the amount of antibody or fragment thereof administered on day three of the dosing regimen is more than about 0.5 mg less than the amount administered on day two.

In certain embodiments, the amount of anti-CD3 antibody or antigen-binding fragment thereof administered on day four of a given dosing regimen is less than about 0.55 mg greater than the amount of anti-CD3 antibody or fragment thereof administered on day three of the dosing regimen. For example, the amount of antibody or fragment thereof administered on day four of the dosing regimen may be less than about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day three. In certain embodiments, the amount of antibody or fragment thereof administered on day four of the dosing regimen is about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day three. In certain embodiments, the amount of antibody or fragment thereof administered on day four of the dosing regimen is about equal to the amount administered on day three. For example, the amount of antibody or fragment thereof administered on day four of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day three. In certain embodiments, the amount of antibody or fragment thereof administered on day four of the dosing regimen is more than about 0.5 mg less than the amount administered on day three.

In certain embodiments, the amount of anti-CD3 antibody or antigen-binding fragment thereof administered on day five of a given dosing regimen is less than about 0.6 mg greater than the amount of anti-CD3 antibody or fragment thereof administered on day four of the dosing regimen. For example, the amount of antibody or fragment thereof administered on day five of the dosing regimen may be less than about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on day four. In certain embodiments, the amount of antibody or fragment thereof administered on day five of the dosing regimen is about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on day four. In certain embodiments, the amount of antibody or fragment thereof administered on day five of the dosing regimen is about equal to the amount administered on day four. In certain embodiments, the amount of antibody or fragment thereof administered on day five of the dosing regimen is less than the amount administered on day four. For example, the amount of antibody or fragment thereof administered on day five of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on day four. In certain embodiments, the amount of antibody or fragment thereof administered on day five of the dosing regimen is more than about 0.5 mg less than the amount administered on day four.

In certain embodiments, the amount of anti-CD3 antibody or antigen-binding fragment thereof administered on day five of a given dosing regimen is at least about 0.5 mg. For example, the amount of antibody or fragment thereof administered on day five of a given dosing regimen can be at least about 0.5 mg, at least about 0.55 mg, at least about 0.6 mg, at least about 0.65 mg, at least about 0.7 mg, at least about 0.75 mg, at least about 0.8 mg, at least about 0.85 mg, at least about 0.9 mg, at least about 0.95 mg, at least about 1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.5 mg, at least about 3 mg, at least about 3.5 mg, at least about 4 mg, at least about 4.5 mg, at least about 5 mg, or higher. In certain embodiments, the amount of antibody or fragment thereof administered on day five of a given dosing regimen is about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg, about 3.5 mg, about 4 mg, about 4.5 mg, about 5 mg, or higher.

In certain embodiments, an anti-CD3 antibody or antigen-binding fragment thereof is administered according to the following dosing regimen: about 0.1 mg on day one, about 0.2 mg on day two, about 0.3 mg on day three, and about 0.5 mg on each of days four through eight. In certain embodiments, an anti-CD3 antibody or fragment thereof is administered according to the following dosing regimen: about 0.1 mg on day one, about 0.2 mg on day two, about 0.3 mg on day three, and about 0.75 mg on each of days four through eight. In certain embodiments, an anti-CD3 antibody or fragment thereof is administered according to the following dosing regimen: about 0.1 mg on day one, about 0.2 mg on day two, about 0.3 mg on day three, about 0.75 mg day four, about 1.0 mg on day five, about 1.25 mg on day six, about 1.5 mg on day seven, and about 1.75 mg on day eight. In certain embodiments, an anti-CD3 antibody or fragment thereof is administered according to the following dosing regimen: about 0.1 mg on day one, about 0.2 mg on day two, about 0.3 mg on day three, about 0.75 mg day four, about 1.0 mg on day five, about 1.25 mg on day six, about 1.5 mg on day seven, and about 3.5 mg on day eight. In certain embodiments, an anti-CD3 antibody or fragment thereof is administered according to the following dosing regimen: about 0.1 mg on day one, about 0.3 mg on day two, about 0.5 mg on day three, about 0.9 mg day four, and about 1.3 mg on day five. In certain embodiments, an anti-CD3 antibody or fragment thereof is administered according to the following dosing regimen: about 0.2 mg on day one, about 0.4 mg on day two, about 0.6 mg on day three, about 0.8 mg day four, and about 1.1 mg on day five.

In certain embodiments, the antibody or antigen-binding fragment thereof is administered in multiple doses on one or more days of any of the above-described dosing regimens. For example, the antibody or fragment thereof may be administered in two doses on day eight of a given dosing regimen to achieve a total daily dose of 3.75 mg or more.

In certain embodiments, the total amount of the anti-CD3 antibody or antigen-binding fragment thereof administered to the patient does not exceed 300 μg/kg when administered intravenously, and when administered other than intravenously, the total amount administered does not exceed the bioequivalent of intravenous administration of 300 μg/kg.

In certain embodiments, the total amount of anti-CD3 antibody or antigen-binding fragment thereof administered over the course of a dosing regimen is no greater than about 21 mg. For example, the total amount of antibody or fragment thereof administered to a patient over the course of a dosing regimen may no greater than about 21 mg, about 20 mg, about 19 mg, about 18 mg, about 17 mg, about 16 mg, about 15 mg, about 14 mg, about 13 mg, about 12 mg, about 11.5 mg, about 11 mg, about 10.5 mg, about 10 mg, about 9.5 mg, about 9 mg, about 8.5 mg, about 8 mg, about 7.5 mg, about 7 mg, about 6.5 mg, about 6 mg, about 5.5 mg, about 5 mg, about 4.5 mg, about 4 mg, about 3.9 mg, about 3.8 mg, about 3.7 mg, about 3.6 mg, about 3.5 mg, about 3.4 mg, about 3.3 mg, about 3.2 mg, about 3.1 mg, about 3 mg, about 2.9 mg, about 2.8 mg, about 2.7 mg, about 2.6 mg, about 2.5 mg, about 2.4 mg, about 2.3 mg, about 2.2 mg, about 2.1 mg, about 2 mg, about 1.9 mg, about 1.8 mg, about 1.7 mg, about 1.6 mg, about 1.5 mg, 1.4 mg, 1.3 mg, 1.2 mg, 1.1 mg, 1 mg, or less. In certain embodiments, the total amount of anti-CD3 antibody or fragment thereof administered over the course of a dosing regimen is no greater than about 8.6 mg. In certain embodiments, the total amount of anti-CD3 antibody or fragment thereof administered over the course of a dosing regimen is no greater than about 6.85 mg. In certain embodiments, the total amount of anti-CD3 antibody or fragment thereof administered over the course of a dosing regimen is no greater than about 3.1 mg.

Any method of administration may be used to administer anti-CD3 antibodies or antigen-binding fragments thereof to a patient. In certain embodiments, an anti-CD3 antibody or fragment thereof is administered to a patient intravenously. In certain embodiments, an anti-CD3 antibody or fragment thereof is administered to a patient by a route other than an intravenous route. For example, the antibody or fragment thereof may be administered to a patient orally, rectally, intramuscularly, intranasally, subcutaneously, intraocularly, transdermally, by direct injection into an affected organ or tissue site, or inhaled. In other embodiments, the antibody or fragment thereof is administered as a continuous infusion (e.g., by a microinfusion pump or slow-release patch). In some embodiments, the patient self-administers the antibody or fragment thereof. Those of ordinary skill in the art will be aware of suitable routes of administration and will be able to adapt such routes of administration to any of the dosing regimens disclosed herein.

In certain embodiments, an anti-CD3 antibody or antigen-binding fragment thereof is administered in a single daily dose on at least one day of a dosing regimen. In certain embodiments, an anti-CD3 antibody or fragment thereof is administered in a single daily dose on each day of a dosing regimen. A single daily dose of antibody or fragment thereof may be administered over a relatively short period of time, e.g., within a period of less than about fifteen minutes. Such embodiments minimize the hospital time and inconvenience to a patient. Alternatively, a single daily dose may be administered to a patient over a longer period of time, e.g., over a period of greater than fifteen minutes. For example, a single daily dose may be administered to a patient over a period of fifteen minutes, thirty minutes, forty-five minutes, one hour, two hours, three hours, four hours, five hours, six hours, seven hours, eight hours, nine hours, ten hours, eleven hours, twelve hours, or more. Such embodiments are useful when, for example, the patient experiences adverse side effects from administering an antibody or fragment thereof over a relatively short period of time. Administration of an antibody or fragment thereof to a patient over a period of time may be accomplished in any of a variety of ways such as, without limitation, intravenous administration.

In certain embodiments, an anti-CD3 antibody or antigen-binding fragment thereof is administered more than once a day on at least one day of a dosing regimen. In certain embodiments, an anti-CD3 antibody or fragment thereof is administered more than once a day on each day of a dosing regimen. For example, an antibody or fragment thereof can be administered twice, three times or four times on at least one day, or each day, of a dosing regimen. In such embodiments, there will typically be an interval between daily doses. For example, the interval between daily doses can be 1 hour, 2 hours, three hours, four hours, five hours, six hours, seven hours, eight hours, nine hours, ten hours, eleven hours, twelve hours, or more. Such embodiments are useful when, for example, the patient experiences adverse side effects from administration of the antibody or fragment thereof in a single daily dose.

In certain embodiments, methods disclosed herein can be used to treat immune-related diseases in nonhuman animals. Doses and methods of administration may be selected in accordance with known principles of veterinary pharmacology and medicine. Guidance may be found, for example, in Adams, R. (ed.), Veterinary Pharmacology and Therapeutics, 8.sup.th edition, Iowa State University Press; ISBN: 0813817439; 2001.

### Ramped Dosing Regimens

Any of the dosing regimens disclosed in the “Exemplary Dosing Regimens” section above, may contain a ramping period. “Ramp” or “ramping period” as the terms are used herein refer to a portion of a dosing regimen over which the amount of antibody or fragment administered increases from a ramp day at the beginning of the ramping period to a ramp day at the end of the ramping period. “Ramp day” as the term is used herein refers to a given day within the ramping period. In certain embodiments, the ramping period is at least two days, e.g., at least three days, at least four days, at least five days, at least six days, at least seven days, at least eight days, at least nine days, at least ten days, at least eleven days, at least twelve days, at least thirteen days, at least fourteen days, or more. In certain embodiments, the ramping period is at most fourteen days, e.g., at most thirteen days, at most twelve days, at most eleven days, at most ten days, at most nine days, at most eight days, at most seven days, at most six days, at most five days, at most four days, at most three days, or fewer. In certain embodiments, the ramping period is two days, three days, four days, five days, six days, seven days, eight days, nine days, ten days, eleven days, twelve days, thirteen days, fourteen days or more. In certain embodiments, the ramping period is four days.

Methods disclosed herein that include a ramping period permit administration of higher cumulative doses of the anti-CD3 antibody or antigen-binding fragment with decreased pro-inflammatory cytokine release and immunogenicity, and with minimal to no perturbation of Epstein Barr Virus immunity. In certain embodiments, methods disclosed herein that include a ramping period facilitate higher individual doses later in a dosing regimen than would be possible with traditional dosing regimens.

In general a ramping period comprises the following characteristics: the antibody or antigen-binding fragment is administered in an amount greater than about 0.1 mg and less than about 0.5 mg on ramp day one; the amount of antibody or fragment administered on ramp day two is less than about 0.5 mg greater than the amount of antibody or fragment administered on ramp day one; the amount of antibody or fragment administered on ramp day three is less than about 0.55 mg greater than the amount of antibody or fragment administered on ramp day two; the amount of antibody or fragment administered on ramp day four is less than about 0.6 mg greater than the amount of antibody or fragment administered on ramp day three; the amount of antibody or fragment administered on ramp day four is more than 0.3 mg greater than the amount of antibody or fragment administered on ramp day one; and the amount of antibody or fragment administered at least one ramp day is at least about 0.5 mg.

In certain embodiments, an anti-CD3 antibody or antigen-binding fragment is administered in an amount greater than about 0.1 mg and less than about 0.5 mg on ramp day one. For example, an antibody or fragment may be administered in an amount of about 0.1 mg, 0.15 mg, 0.2 mg, 0.25 mg, 0.3 mg, 0.35 mg, 0.4 mg, 0.45 mg, or 0.5 mg on ramp day one.

In certain embodiments, the amount of anti-CD3 antibody or antigen-binding fragment administered increases between ramp day one and ramp day four of a given dosing regimen. In certain embodiments, the amount of increase between ramp day one and ramp day four is more than about 0.3 mg. For example, the amount of anti-CD3 antibody or fragment administered may increase more than about 0.3 mg, more than about 0.35 mg, more than about 0.4 mg, more than about 0.45 mg, more than about 0.5 mg, more than about 0.55 mg, more than about 0.6 mg, more than about 0.65 mg, more than about 0.7 mg, more than about 0.75 mg, more than about 0.8 mg, more than about 0.85 mg, more than about 0.9 mg, more than about 0.95 mg, more than about 1.0 mg, more than about 1.1 mg, more than about 1.2 mg, more than about 1.3 mg, more than about 1.4 mg, more than about 1.5 mg, more than about 1.6 mg, more than about 1.7 mg, more than about 1.8 mg, more than about 1.9 mg, more than about 2 mg, more than about 2.5 mg, more than about 3 mg, more than about 3.5 mg, more than about 4 mg, more than about 4.5 mg, more than about 5 mg, or more.

In certain embodiments, the amount of anti-CD3 antibody or antigen-binding fragment administered increases on each day between ramp day one and ramp day four of a given dosing regimen such that the total increase between ramp day one and ramp day four is more than about 0.3 mg. In certain embodiments, the amount of anti-CD3 antibody or fragment administered between ramp day one and ramp day four of a given dosing regimen increases by more than about 0.3 mg, but the amount of anti-CD3 antibody or fragment administered does not increase on each day. For example, the amount of antibody or fragment administered may remain constant or even decrease between, e.g., ramp day one and ramp day two, ramp day two and ramp day three, or ramp day three and ramp day four, but the total amount nevertheless increases by more than about 0.3 mg between ramp day one and ramp day four.

In certain embodiments, the amount of anti-CD3 antibody or antigen-binding fragment administered on ramp day two of a given dosing regimen is less than about 0.5 mg greater than the amount of anti-CD3 antibody or fragment administered on ramp day one of the dosing regimen. For example, the amount of antibody or fragment administered on ramp day two of the dosing regimen may be less than about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day one. In certain embodiments, the amount of antibody or fragment administered on ramp day two of the dosing regimen is about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day one. In certain embodiments, the amount of antibody or fragment administered on ramp day two of the dosing regimen is about equal to the amount administered on ramp day one. In certain embodiments, the amount of antibody or fragment administered on ramp day two of the dosing regimen is less than the amount administered on ramp day one. For example, the amount of antibody or fragment administered on ramp day two of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day one. In certain embodiments, the amount of antibody or fragment administered on ramp day two of the dosing regimen is more than about 0.5 mg less than the amount administered on ramp day one.

In certain embodiments, the amount of anti-CD3 antibody or antigen-binding fragment administered on ramp day three of a given dosing regimen is less than about 0.55 mg greater than the amount of anti-CD3 antibody or fragment administered on ramp day two of the dosing regimen. For example, the amount of antibody or fragment administered on ramp day three of the dosing regimen may be less than about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day two. In certain embodiments, the amount of antibody or fragment administered on ramp day three of the dosing regimen is about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day two.

In certain embodiments, the amount of antibody or fragment administered on ramp day three of the dosing regimen is about equal to the amount administered on ramp day two. For example, the amount of antibody or fragment administered on ramp day three of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day two. In certain embodiments, the amount of antibody or fragment administered on ramp day three of the dosing regimen is more than about 0.5 mg less than the amount administered on ramp day two.

In certain embodiments, the amount of anti-CD3 antibody or antigen-binding fragment administered on ramp day four of a given dosing regimen is less than about 0.6 mg greater than the amount of anti-CD3 antibody or fragment administered on ramp day three of the dosing regimen. For example, the amount of antibody or fragment administered on ramp day four of the dosing regimen may be less than about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater, or less than on ramp day three. In certain embodiments, the amount of antibody or fragment administered on ramp day four of the dosing regimen is about 0.6 mg greater, about 0.55 mg greater, about 0.5 mg greater, about 0.45 mg greater, about 0.4 mg greater, about 0.35 mg greater, about 0.3 mg greater, about 0.25 mg greater, about 0.2 mg greater, about 0.15 mg greater, about 0.1 mg greater, about 0.09 mg greater, about 0.08 mg greater, about 0.07 mg greater, about 0.06 mg greater, about 0.05 mg greater, about 0.04 mg greater, about 0.03 mg greater, about 0.02 mg greater, about 0.01 mg greater than on ramp day three. In certain embodiments, the amount of antibody or fragment administered on ramp day four of the dosing regimen is about equal to the amount administered on ramp day three. In certain embodiments, the amount of antibody or fragment administered on ramp day four of the dosing regimen is less than the amount administered on ramp day three. For example, the amount of antibody or fragment administered on ramp day four of the dosing regimen may be about 0.01 mg less, about 0.02 mg less, about 0.03 mg less, about 0.04 mg less, about 0.05 mg less, about 0.06 mg less, about 0.07 mg less, about 0.08 mg less, about 0.09 mg less, about 0.1 mg less, about 0.15 mg less, about 0.2 mg less, about 0.25 mg less, about 0.3 mg less, about 0.35 mg less, about 0.4 mg less, about 0.45 mg less, about 0.5 mg less, than the amount administered on ramp day three. In certain embodiments, the amount of antibody or fragment administered on ramp day four of the dosing regimen is more than about 0.5 mg less than the amount administered on ramp day three.

In certain embodiments, the amount of anti-CD3 antibody or antigen-binding fragment administered on ramp day four of a given dosing regimen is at least about 0.5 mg. For example, the amount of antibody or fragment administered on ramp day four of a given dosing regimen can be at least about 0.5 mg, at least about 0.55 mg, at least about 0.6 mg, at least about 0.65 mg, at least about 0.7 mg, at least about 0.75 mg, at least about 0.8 mg, at least about 0.85 mg, at least about 0.9 mg, at least about 0.95 mg, at least about 1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.5 mg, at least about 3 mg, at least about 3.5 mg, at least about 4 mg, at least about 4.5 mg, at least about 5 mg, or higher. In certain embodiments, the amount of antibody or fragment administered on ramp day four of a given dosing regimen is about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg, about 3.5 mg, about 4 mg, about 4.5 mg, about 5 mg, or higher.

In certain embodiments, an anti-CD3 antibody or antigen-binding fragment is administered on at least one pre-ramp day prior to ramp day one. For example, an antibody or fragment may be administered on one, two, three, four, five, six, seven, eight, nine, ten, or more pre-ramp days prior to ramp day one. In certain embodiments, the amount of antibody or fragment administered on at least one pre-ramp day does not exceed 0.3 mg, e.g., does not exceed 0.25 mg, 0.2 mg, 0.15 mg, 0.1 mg, 0.05 mg, or less. In certain embodiments, the amount of antibody or fragment administered on at least one pre-ramp day is about 0.1 mg. In certain embodiments, the amount of antibody or fragment administered on at least one pre-ramp day is about 0.2 mg. In certain embodiments, the amount of antibody or fragment administered on at least one pre-ramp day is about 0.3 mg.

### Dosing Regimens Based on Molecular Weight of Antibody or Fragment

In certain embodiments, anti-CD3 antibodies or antigen-binding fragments thereof can be administered without regard to the molecular weight of the antibody or fragment, or to the number of antigen binding sites in a given antibody or fragment. For example, any of the dosing regimens described above can be administered to patient regardless of molecular weight or number of antigen binding sites.

“Molecular weight” is a term and concept well known to those of ordinary skill in the art. The molecular weight of a compound or composition is the weight of one molecule of the compound or composition, relative to the unified atomic mass unit u (defined as 1/12 the mass of one molecule of the carbon-12 isotope). A compound or composition having a given molecular weight can also be quantified by molar mass, which has a numerical value that is the average molecular weights of the molecules in the compound or composition multiplied by Avogadro's constant (approximately 6.022×10²³). Molar mass is expressed in terms of grams per mole.

Antibodies vary in molecular weight based on, for example, the length and amino acid composition of the heavy and light chain polypeptide sequences that make up the protein part of the antibody. Moreover, as is known to those of ordinary skill in the art, the molecular weight of an antibody varies according to the extent of post-translational modification the antibody undergoes. For example, antibodies are often subjected to glycosylation, in which one or more carbohydrate moieties are covalently attached to either the heavy or light chain polypeptide sequence. Even amongst a population of antibodies with identical heavy and light chain polypeptide sequences, the extent and type of glycosylation can vary. The molecular weights of many antibodies are known in the art. Additionally, the molecular weight of a particular antibody can be empirically determined with any of a variety of tools known to those of ordinary skill in the art such as, without limitation, mass spectrometry. Determining the molecular weight of any particular antibody is within the abilities of those of ordinary skill in the art.

Antibody fragments also vary in molecular weight based on, for example, the length and amino acid composition of the heavy and light chain polypeptide sequences and post-translational glycosylation patterns. Certain antibody fragments, such as without limitation, Fab fragment, F(ab′)₂ fragments, and scFv fragments, are typically of a much lower molecular weight that that of an antibody that includes both heavy and light polypeptide chains. As with full-length antibodies, the molecular weight of particular antibody fragment can be empirically determined with any of a variety of tools known to those of ordinary skill in the art such as, without limitation, mass spectrometry, and is within the abilities of those of ordinary skill in the art

In certain embodiments, anti-CD3 antibodies or antigen-binding fragments thereof can be administered based on the molecular weight of that antibody or fragment. Such molecular weight-based dosing regimens can be useful when, for example, a practitioner desires to administer a dosing regimen of a particular antibody or fragment, the molecular weight of which differs from the molecular weight of another antibody or fragment thereof used in an identical or similar dosing regimen. In certain embodiments, by calibrating the amount of antibody or fragment thereof administered based on the molecular weight of the particular antibody or fragment, a more uniform molar amount of antibody or fragment thereof can be administered to a patient.

For example, otelixizumab has an average molecular weight of approximately 145 kDa. Thus, if a particular dosing regimen calls for 0.1 mg of antibody to be administered to a patient on a particular day, the patient can be administered approximately 6.90×10⁻¹⁰ moles of otelixizumab. Doses of different antibodies or fragments thereof can be similarly calculated based on the molecular weight of those antibodies or fragments thereof. In certain embodiments, an antibody or fragment thereof with a larger molecular weight is administered to the patient in a greater per-weight amount. In other embodiments, an antibody or fragment thereof with a smaller molecular weight is administered to the patient in a lower per-weight amount.

As another non-limiting example, the following dosing schedule can be used: 0.1 mg on day 1, 0.3 mg on day 2, 0.5 mg on day 3, 0.9 mg on day 4, and 1.3 mg on day 5. Based on a reference antibody with a molecular weight of 145 kDa, for example, one can administer such a dosing schedule to a patient based on the specific molecular weight of the antibody of fragment to be administered as follows: 6.90×10⁻¹⁰ moles on day 1, 2.07×10⁻⁹ moles on day 2, 3.45×10⁻⁹ moles on day 3, 6.21×10⁻⁹ moles on day 4, and 8.96×10 moles on day 5. Those of ordinary skill in the art can calculate the molar amounts of antibody or fragment thereof to be given for any of the dosing regimens disclosed herein.

In certain embodiments, anti-CD3 antibodies or antigen-binding fragments thereof can be administered based on the number of antigen binding sites present on the antibody or fragment. As is known to those of ordinary skill in the art, a whole antibody includes two distinct antigen binding sites which are located in the hypervariable regions (also known as the complementarity determining region or CDR) of the antibody. The antigen binding sites of whole antibodies are formed by an interaction between the variable regions of the heavy and light chains. Each antigen binding site is capable of binding one antigen. For example, whole IgG antibodies are capable of binding two antigens. Certain antibody fragments also can include two antigen binding sites. For example, a F(ab′)₂ fragment lacks the constant region of a whole antibody, yet retains two antigen binding sites. Certain antibody fragments include only a single antigen binding site. For example, Fab fragments and scFv fragments lack the constant region of a whole antibody, and include only a single antigen binding site. Those of ordinary skill in the art will be aware of various antibody fragments, and will know how many antigen binding sites each fragment contains.

In certain embodiments, anti-CD3 antibodies or antigen-binding fragments thereof can be administered based on the number of antigen binding sites present in a given antibody or fragment. Such antigen binding site-based dosing regimens can be useful when, for example, a practitioner desires to administer a dosing regimen of a particular antibody or fragment thereof that includes a different number of antigen binding sites as compared to the number of antigen binding sites of another antibody or fragment thereof used in an identical or similar dosing regimen. In certain embodiments, by calibrating the amount of antibody or fragment thereof administered during a dosing regimen based on the number of antigen binding sites that antibody or fragment thereof possesses, a more uniform number of antigen binding sites can be administered to a patient.

For example, otelixizumab possesses two antigen binding sites per molecule. Thus, if a particular dosing regimen calls for 0.1 mg of antibody to be administered to a patient on a particular day, the patient can be administered approximately 0.1 mg of otelixizumab, or 0.2 mg of an antibody or fragment thereof that possesses only one antigen binding site per molecule. Doses of different antibodies or fragments thereof can be similarly calculated based on the number of antigen binding sites those antibodies or fragments thereof possess. In certain embodiments, an antibody or fragment thereof with one antigen binding site per molecule is administered to the patient in a greater amount than an antibody with two or more antigen binding sites per molecule. In other embodiments, an antibody or fragment thereof with two or more antigen binding sites per molecule is administered to the patient in a lower amount than an antibody with only one antigen binding site per molecule.

As another non-limiting example, the following dosing schedule can be used: 0.1 mg on day 1, 0.3 mg on day 2, 0.5 mg on day 3, 0.9 mg on day 4, and 1.3 mg on day 5. Based on a reference antibody having two antigen binding sites, for example, one can administer an antibody or fragment thereof having only one antigen binding site to a patient according to the dosing schedule as follows: 0.2 mg on day 1, 0.6 mg on day 2, 1.0 mg on day 3, 1.8 mg on day 4, and 2.6 mg on day 5. Those of ordinary skill in the art can calculate the amount of antibody or fragment thereof to be given for any of the dosing regimens disclosed herein based on the number of antigen binding sites the antibody or fragment thereof possesses.

Those of ordinary skill in the art will be able to calculate weight-based and body surface-based dosing regimens that correspond to any of the variety of dosing regimens disclosed in the present specification, and will be able to administer such dosing regimens to a patient.

Moreover, those of ordinary skill in the art will be able to choose a dosing regimen of an particular antibody or antigen-binding fragment thereof based on a combination of one or more of: the body weight of a patient, the body surface area of a patient, the molecular weight of the antibody or fragment, the number of target antigens in a given patient's body, and the number of antigen binding sites of the antibody or fragment. For example, a patient that weighs more than 80 kg can be administered an antibody or fragment thereof that possesses only one antigen binding site. In such an example, a larger amount of antibody or fragment thereof can be administered to account for (1) the patient's increased weight, and (2) the fact that the antibody or fragment thereof has fewer antigen binding sites than a bivalent whole antibody. Upon reading the present specification, those of ordinary skill in the art will be able to administer an antibody or fragment thereof to a patient in a dosing regimen specifically tailored to the physical characteristics of the patient and/or the molecular properties of the antibody or fragment.

### PK/PD Parameters

Subjects administered any of the presently disclosed dosing regimens may experience one or more immunoregulatory effects, such as one or more of those described in this section. The presently disclosed methods of treating immune-related diseases are not limited in any way by any particular mechanism of action. Nevertheless, a number of pharmacodynamic (PD) effects of treating T cells with reduced Fcγ receptor-binding anti-CD3 antibodies or CD3-binding fragments thereof according to methods disclosed herein, are observable. For convenience these reduced Fcγ receptor-binding anti-CD3 antibodies and CD3-binding fragments are referred to in this PK/PD Parameters section as “CD3-binding agents”

In broad terms, the immunoregulatory effects seen after administration of CD3-binding agents can be divided into two phases that can overlap to some degree. Thus in the initial early phase (from an hour up to about 14 days) following exposure of T cells (CD4+ and CD8+) to such CD3-binding agents (in vivo and in vitro) immunoregulatory effects that occur include down-modulation of TCR/CD3 complexes on the surfaces of the T cells, induction of T cell anergy or hyporesponsiveness to antigen, induction of apoptosis of T cells, and a decrease in the numbers of T cells (CD4+ T cells and CD8+ T cells). With respect to in vitro exposures, solid or gel substrate (e.g., tissue culture well bottom or agarose bead)-bound anti-CD3 antibodies, and CD3-binding fragments thereof, that have reduced ability to bind Fcγ receptors do not qualify as “CD3-binding agents” (as defined above) in this substrate-bound form since they act in the same way as anti-CD3 antibodies with normal, wild-type Fcγ receptor binding activity in the presence of Fcγ receptor expressing cell. In the later phase (from one days to 16 weeks or more) after the exposure, the levels of immunosuppressive CD4+ T cells (Tregs) expressing both cell surface CD25 (i.e., CD25+) and the FoxP3 transcription factor (FoxP3+) are found to increase. Notably no increase in CD8+, CD25+, FoxP3+cells is seen. Some or all of these events are interrelated.

T cells that undergo apoptosis as a result of exposure to CD3-binding agents, which is generally by the Fas/Fas ligand pathway, are those that are activated by antigen prior to the exposure (and are progressing through the cell cycle) and are not resting T cells. T cells in the S-G2 phase of the cell cycle are particularly sensitive to this type of apoptosis. The decreases in the numbers of CD4+and CD8+ T cells that are seen in the first phase appear to reflect retrafficking of T cells (e.g., from the blood to lymphoid tissue and/or target organs) and, possibly, to a relatively small extent, the above-described apoptosis.

The initial decrease of antigen responsiveness of T cells that have not undergone apoptosis is to some degree correlated with CD3/TCR down-modulation on the surface of the T cells. Nevertheless, there are conditions under which drastically reduced antigen responsiveness in the T cells is observed in the face of significant levels of cell surface TCR (see, e.g., Schwartz (2003) Annu. Rev. Immunol. 21:305-334). These findings indicate that, while antigen hyporesponsiveness in the T cells exposed to TCR/CD3-binding agents is due at least in part to down-modulation of CD3/TCR complexes, it is likely also due to the other effects such as active CD3/TCR-mediated anergy induction. It is also clear that, while transient exposure of T cells to lower doses of CD3-binding agents results in transient anergy or antigen hyporesponsiveness of T cells and cell-surface CD3/TCR down-modulation (with full recovery within less than 24 hours of exposure), longer exposure to somewhat higher doses results in much longer, if not permanent, anergy or antigen hyporesponsiveness (see, e.g., Anasetti et al. (1990) J. Exp. Med. 172:1691-1700; and Forman et al. (2009) Immune Privilege and Tolerance-Therapeutic Antibody Approaches. In: Recombinant Antibodies for Immunotherapy, M. Little, Ed., Cambridge University Press, pp. 350-369). Down-modulation of CD3/TCR in response to CD3-binding agents seems to be largely due to internalization of CD3-binding agent:CD3/TCR complexes rather than masking of the CD3/TCR complex by the binding agent.

The transient effects (anergy or antigen hyporesponsiveness of T cells and cell-surface CD3/TCR down-modulation) indicated above to occur as a result of exposure to CD3-binding agents are seen even when repeated doses (e.g., on a daily basis) are administered. The anergy/antigen hyporesponsiveness and cell-surface CD3/TCR down-modulation occur after the first administration but the levels of both return to normal (i.e., the levels prior to the first administration) by the time of the second administration. The same effect is seen after all subsequent administrations unless much higher doses are administered and/or the cells are exposed to the CD3-binding agent for a much longer time. This pattern of decrease and increase in these parameters is referred to herein as a “saw tooth pattern”. Interestingly, with respect to the levels of both CD4+and CD8+ T cells, while a saw tooth pattern is seen, it is accompanied by an overall decrease in the total numbers of the cells during the course of the CD3-binding agent (see, e.g., Examples 2-4). Thus, after each successive administration, the rebound seen after the initial decrease in cell numbers after an administration is to a lower level than after the immediately previous administration.

It is likely that the induction of anergy or antigen hyporesponsiveness in T cells by these CD3-binding agents that, as indicated above have reduced ability to bind to Fcy receptors, is analogous to that of altered peptide ligands (APL) (see, e.g.: Sloan-Lancaster et al. (1993) Nature 363:156-159; Sloan-Lancaster et al. (1994) Cell 79:913-922; and Madrenas et al. (1995) 267:515-518) that result in weak or incomplete activation of T cells. One likely mechanism of CD3-binding agent-induced anergy induction involves reduction in the relative proportion of cell surface TCR/CD3 multimeric clusters to cell-surface monovalent TCR/CD3 complexes. It has been shown that TCR/CD3 complexes on T cells occur as both monovalent units and multivalent clusters, the latter existing in a wide range multiplicities (from two to greater than 20 TCR/CD3 monomers) and the monomer in each case containing a TCR α and β chain (or a TCR γ and δ chain) and one CD3δ, two CD3ε, one CD3γ, and two CD3ζ chains (see, e.g.: Alarcón et al. (2006) EMBO Reports 7: 490-495; and Schamel et al. (2005) J. Exp. Med. 202(4): 493-503). Thus, by exposing T cells to increasing concentrations of CD3-binding agents, the relative level of higher avidity CD3/TCR multimer clusters is decreased, leaving behind the lower avidity CD3/TCR monovalent units and thereby reducing the potential CD3/TCR signal strength and T cell responsiveness. The lower the level of multimers left after exposure, the longer it will take a particular T cell to recover fully activating signal strength responsiveness by synthesizing new multimers, recirculating down modulated multimers back to the surface, and/or converting monomeric units into multivalent complexes. This phenomenon could also explain the “conditioning” effect observed when an animal (e.g., a human) is administered a dosing regimen that includes a ramping period, as disclosed herein. Without wishing to be bound by theory, it is hypothesized that conditioning may result from the lower ramping doses being sufficient to modulate but not activate, so that when subsequent larger activating doses are given later in a dosing regimen, the signal strength is weak or incomplete leading to relative low responses and anergy. At some critical concentration of CD3-binding agent and/or length of exposure of the T cell to the CD3-binding agent, the T cell will be rendered anergic for an extremely long time, possibly for its life time. The relative susceptibility of T cells to anergy induction would depend on a number of factors, including the relative number of multimeric CD3/TCR clusters to monovalent CD3/TCR units and the relative number of monomeric units in the clusters.

The induction of CD4+ Tregs that occurs later in the response of CD4+ T cells to CD3-binding agents is likely to be relatively more important in the long-term beneficial effects of CD3-binding agents to immune-related (especially T cell-mediate) diseases, including autoimmune diseases such as type 1 diabetes (insulin-dependent diabetes mellitus (IDDM)), psoriasis, multiple sclerosis, and rheumatoid arthritis. Their induction very likely involves factors (e.g., transforming growth factor β (TGF-β)) produced by, and/or cell-cell interactions with, the hyporesponsive (or completely anergized) T cells described above as well as antigen presenting cells such as dendritic cells, and does not necessarily require contacting of the Treg precursor cells themselves with a CD3-binding agent.

In light of the above considerations, methods of inducing hyporesponsiveness and/or anergy, apoptosis, decreases in the numbers of CD4+and CD8+ T cells, cell surface TCR/CD3 down-modulation, and relative level of multivalent TCR/CD3 clusters (as compared to monovalent TCR/CD3 units) in target T cells (e.g., CD4+and CD8+ T cells to which the CD3-binding agents bind) down-modulation are provided. Also provided are methods for inducing CD4+, CD25+, FoxP3+ Treg cells. All these methods involve exposing target T cells to CD3-binding agents either in vivo or in vitro. Where the exposing is in vitro, the CD3-binding agents are in solution rather than bound to a solid or gel substrate (see above). In the induction of Treg cells, the precursor of the Treg can be, but is not necessarily, a target T cell (as the term is used above). Moreover, CD3-binding agents can bind to established CD4+CD25+FoxP3+ Tregs and thereby enhance their suppressive activity. The dosing and scheduling regimens and methods of administration for performing in vivo exposures can be any of those disclosed herein, as are the subjects to which the methods can be applied.

While the target T cells are more commonly CD4+ T cells, it is understood that they can also be CD8+ T cells. Moreover, CD4+and CD8+effector T cells (e.g., pathogenic T cells involved in a disease process) are subject to the suppressive activity of CD4+CD25+FoxP3+ Tregs. However, it is understood that CD25+, FoxP3+ T regs per se are CD4+and not CD8+. The TCR/CD3 down-modulation can be complete (100%) or partial (e.g., at least or not greater than: 10%:20%; 30%; 40%; 50%; 60%; 70%; 80%; 90%; 95%; or 98%). The down-modulation of the number of multivalent CD3/TCR clusters (i.e., units containing more than one CD3/TCR complex unit (see above)) can be similarly complete or partial. An anergic T cell is one that has substantially no responsiveness (i.e., less than 5%) as compared to the responsiveness that T cell would have had without exposure to a CD3-binding agent or the average responsiveness of T cells having the same CD4/CD8 cell surface marker as well as other markers known in the art to be associated with pre-exposure, or lack thereof, to antigen. T cells can be naive T cells (i.e., those never pre-exposed to antigen), activated T cells (i.e., T cells exposed to antigen and displaying any of a variety of T cell activities, e.g., proliferation, cytotoxic activity, and cytokine production), or memory T cells (i.e., T cells exposed to antigen and having an enhanced ability to respond to the same antigen and not necessarily displaying an activated cell phenotype. Cell surface markers positively (+) and negatively (−) associated with naive T cells include: CD45RA+, CD26L+, CD45 edited isoforms (CD45RB, CD45RC, CD45RAB, CD45RAC, CD45RBC, CD45RO, CD45R (ABC))−, CD25−, CD44−, and CD69−. Cell surface markers positively (+) associated with activated T cells include: CD25+, CD69+, HLA-DR+, CD38+, and GITR+. Memory T cells fall into three broad categories, which are categorized as follows: central memory T cells (memory stem cells) (T_{CM}) (L-selectin+, chemokine receptor CCR7+, and produce interleukin (IL)-2 (IL-2) but not IL-4 or interferon γ (IFN-γ)); effector memory T cells (T_{EM}) and closely related effector memory T cells RA (T_{EMRA}) (L-selectin-, CCR7−, and produce IL-4 and IFN-γ).

With respect to pharmacokinetic (PK) data, it has been possible to determine PK parameters for a CD3-binding agent of interest (the TRX4 antibody, also known as otelixizumab) using data collected from a number of clinical studies (see Table 1). The serum otelixizumab concentrations versus time were described by a one-compartment model with Michaelis-Menten (MM) saturable elimination:

$$\frac{d {C}_{p}}{d t} = Input / {V}_{d} - {V}_{max} {C}_{p} / ( {K}_{m} + {C}_{p} )$$

$${C}_{p} ( 0 ) = 0$$

where Cₚ is serum concentration of otelixizumab, V_{d} is the volume of distribution, Vₘₐₓ is the capacity of the elimination process, and Kₘ is the affinity constant or the serum otelixizumab concentration at which the elimination rate attains one-half of Vₘₐₓ.

TABLE 1
Clinical Studies of Otelixizumab Included in PK Analysis

| | Group or | | | Number |
|---|---|---|---|---|
| Study | Cohort | Doses (mg) ^{a} | Disease ^{b} | of subjects |
| I | Group A | 24, 8, 8, 8, 8, 8 | D | 3 |
| | Group B | 8, 8, 8, 8, 8, 8 | D | 37 |
| II | Cohort 1 | 1 | P | 4 |
| | Cohort 2 | 2 | P | 4 |
| | Cohort 3 | 4 | P | 8 |
| III | Cohort 1 | 0.1, 0.1, 0.1 | D | 4 |
| | Cohort 2 | 0.5, 0.5, 0.5 | D | 3 |
| | Cohort 9 | 0.1, 0.3, 0.5 | D | 4 |
| | Cohort 10 | 0.3, 0.5, 1.0 | D | 1 |
| | Cohort A | 0.1, 0.2, 0.3, 0.5 | D | 4 |
| | Cohort A(1/2) | 0.05, 0.1, 0.15, 0.25 | D | 1 |
| | Cohort B | 0.1, 0.2, 0.3, 0.75 | D | 4 |
| | Cohort C | 0.1, 0.2, 0.3, 1.0 | D | 1 |
| | CH1 | 0.1, 0.2, 0.3, 0.5x5 | D | 16 |
| | CH2 | 0.1, 0.2, 0.3, 0.75x5 | D | 18 |
| | CH3 | 0.1, 0.2, 0.3, 0.75, 1, | D | 6 |
| | | 1.25, 1.5, 1.75 | | |

^{a} Doses were given once daily for 1 to 8 days.
^{b} D—Type 1 diabetes; P—Psoriasis.

In Study I (Table 1), otelixizumab was administered 6 times. In Group A, otelixizumab concentrations remained more or less constant over the 6 days of dosing, whereas in group B they increased slightly, suggesting accumulation of the drug.

In Study II (Table 1), otelixizumab was administered only once. Extensive sampling was done over the 24 hours after drug administration. For the 1 mg and 2 mg doses, the concentrations decreased to below the LLQ (lower limit of quantification) in about 0.2 day. For the 4 mg dose, concentrations above LLQ were observed up to 0.8 day. A few subjects showed a biphasic decline with a very rapid first phase.

In Study III (Table 1), otelixizumab was administered daily for up to 8 days. Doses were substantially lower than in Studies I and II, and as a result, most (83%) concentrations were below the LLQ. Due to the limited amount of available PK data, simultaneous analysis of the PK and PD (pharmacodynamic) data was necessary to recover PK profiles. The model building process started with linear PK; however, the individual empirical Bayesian estimates of volume of distribution were dose-dependent, suggesting nonlinearity. Thus, MM elimination was used, leading to substantial improvement in the model. Such kinetic parameters were estimated Kₘ=0.968 μg/mL and Vₘₐₓ=1.35 μg/mL/day. At low concentrations, such as those observed in Study III, otelixizumab was eliminated linearly with elimination rate constant kₑₗ=Vₘₐₓ/Kₘ=1.39 day⁻¹. At high concentrations, elimination was saturated. The V_{d} was estimated as 13.9 L with between-subject variability of about 76%.

Biphasic elimination from serum is usually observed after an intravenous dose of intact antibodies. The intact antibodies rapidly distribute primarily to the highly perfused organs such as kidney, lung and liver. The volume of distribution often equals the plasma volume, 2-3 L. For otelixizumab, the V_{d} of 13.9 L was determined assuming a one-compartment model with MM elimination. This value of V_{d} suggests antibody distribution outside the blood or occurrence of nonspecific binding. Antigen binding can significantly affect the PK of a mAb. Target-mediated drug disposition models were proposed and successfully applied to describe the PK of certain mAbs. In the case of otelixizumab, elimination by binding to TCR/CD3 complexes did not affect its PK. After otelixizumab administration, the TCR/CD3 is down-modulated from T cell surfaces, and the transient trafficking and re-distribution of lymphocytes reduces the total pool of receptors available for binding. However, part of the otelixizumab molecule might bind to the TCR/CD3 and consequently be degraded or redistributed to the peripheral tissue on the T cell surface. The MM elimination was used to approximate observed nonlinearities. The affinity constant (Kₘ=0.968 μg/mL) suggests that PK may become nonlinear at high concentrations such as those observed in Study I. For the dose ranges used in Study III, and to some degree in Study II, the drug is eliminated under linear conditions with a kₑ₁ of 1.39 day⁻¹ and a corresponding half-life of 0.50 day. Intact human IgG₁ exhibits a long half-life of about 3 weeks due to the catabolic protection and recycling by the neonatal Fc receptor (FcRn). For otelixizumab the half-life is much shorter, suggesting that this protection pathway is not active, likely due to the single amino acid substitution in the Fc region which eliminates the only glycosylation site and alters the spatial configuration of the Fc region.

In view of the above PK considerations, in certain embodiments, the present disclosure provides a CD3-binding agent (see above) and a pharmaceutical composition containing it. The CD3-binding agent is an antibody (or CD3-binding fragment thereof) that binds to human CD3 with an affinity constant (Kₘ) of at least 0.968 μg/mL. It can have with a kₑₗ of about 1.39 day⁻¹. Moreover, its half life can be about 0.50 day when administered to a human.

The CD3-binding agent can show non-linear PK at high concentrations (about 8 mg to about 48 mg per day) and linear PK at low concentrations (about 0.1 to about 21 mg per day). Other features of the CD3-binding agent can be those described herein for otelixizumab (TRX4). Moreover the CD3-binding agent can be used in any of the methods and subjects described herein.

In certain embodiments, a pathogenic effect observed in the animal (e.g., on day five) or later of the dosing regimen is decreased or eliminated compared to the pathogenic effect that would be observed that day if the animal were administered a different dosing regimen. “Pathogenic effect” as the term is used herein refers to any adverse effect that results directly or indirectly from a given dosing regimen. A pathogenic effect may be, for example, increased cytokine release, (Epstein Barr Virus) EBV activation, or immunogenicity. In certain embodiments, the different dosing regimen lacks a ramping period. In certain embodiments, the different dosing regimen comprises a dose higher than 0.5 mg on either day one or day two of the different dosing regimen.

In certain embodiments, dosing regimens disclosed herein for treating an immune-related disease (e.g., diabetes) result in a reduced level of release of at least one cytokine compared to an animal that is administered an equivalent dosing regimen of an anti-CD3 antibody or fragment thereof that does not exhibit reduced binding to the Fc (gamma) receptor. For example the release of the at least one cytokine may be reduced by at least 50%, e.g., at least 55%, at least 60%, at least 65%, at least 70%, at least 75%, at least 80%, at least 85%, at least 90%, at least 95%, at least 96%, at least 97%, at least 98%, at least 99%, or more. In certain embodiments, such a cytokine may be a pro-inflammatory cytokine including, but not limited to, IL2, IL6, IL10, IFN-gamma, or tumor necrosis factor alpha (TNF-alpha). Those of ordinary skill in the art will be aware of other pro-inflammatory cytokines, and will be able to measure their levels in a subject that has been administered any of the dosing regimens disclosed herein.

### Pharmaceutical Formulations

Antibodies or antibody fragments described herein may be formulated for delivery by any available route including, but not limited to parenteral (e.g., intravenous), intradermal, subcutaneous, oral, nasal, bronchial, ophthalmic, transdermal (topical), transmucosal, rectal, and vaginal routes. Antibodies or antibody fragments may include a delivery agent (e.g., a cationic polymer, peptide molecular transporter, surfactant, etc., as described above) in combination with a pharmaceutically acceptable carrier. As used herein the term “pharmaceutically acceptable carrier” includes solvents, dispersion media, coatings, antibacterial and antifugal agents, isotonic and absorption delaying agents, and the like, compatible with pharmaceutical administration. Supplementary active compounds can also be incorporated into pharmaceutical formulations comprises an antibody or fragment thereof as described herein.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Pharmaceutical formulations are ideally stable under the conditions of manufacture and storage and should be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the antibody or antibody fragment in the required amount in an appropriate solvent with one or a combination of ingredients enumerated above, as required, followed by filtered sterilization. Generally, dispersions are prepared by incorporating the purified antibody or antibody fragment into a sterile vehicle which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the antibody or antibody fragment can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the antibody or antibody fragment and a delivery agent are preferably delivered in the form of an aerosol spray from a pressured container or dispenser which contains a suitable propellant, e.g., a gas such as carbon dioxide, or a nebulizer. The present disclosure particularly contemplates delivery of the compositions using a nasal spray, inhaler, or other direct delivery to the upper and/or lower airway. Intranasal administration of DNA vaccines directed against influenza viruses has been shown to induce CD8 T cell responses, indicating that at least some cells in the respiratory tract can take up DNA when delivered by this route, and the delivery agents of the invention will enhance cellular uptake. According to certain embodiments, antibody or antibody fragment and a delivery agent are formulated as large porous particles for aerosol administration.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the purified polypeptide or protein and delivery agents are formulated into ointments, salves, gels, or creams as generally known in the art.

In certain embodiments, compositions are prepared with carriers that will protect the antibody or antibody fragment against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active antibody or antibody fragment calculated to produce the desired therapeutic effect in association with the required pharmaceutical carrier.

The antibody or antibody fragment can be administered at various intervals and over different periods of time as required, e.g., one time per week for between about 1 to 10 weeks, between 2 to 8 weeks, between about 3 to 7 weeks, about 4, 5, or 6 weeks, etc. Those of ordinary skill in the art will appreciate that certain factors can influence the dosage and timing required to effectively treat a subject, including but not limited to the severity of the disease or disorder, previous treatments, the general health and/or age of the subject, and other diseases present. Generally, treatment of a subject with an antibody or antibody antigen-binding fragment as described herein can include a single treatment or, in many cases, can include a series of treatments. It is furthermore understood that appropriate doses may depend upon the potency of the antibody or antibody fragment and may optionally be tailored to the particular recipient, for example, through administration of increasing doses until a preselected desired response is achieved. It is understood that the specific dose level for any particular animal subject may depend upon a variety of factors including the activity of the specific polypeptide or protein employed, the age, body weight, general health, gender, and diet of the subject, the time of administration, the route of administration, the rate of excretion, any drug combination, and the degree of expression or activity to be modulated.

Pharmaceutical formulations as described herein can be included in a container, pack, or dispenser together with instructions for administration.

### Combination Therapies

Methods described herein can include administering a course of immunotherapy (e.g, anti-CD3 antibodies and antigen-binding fragments thereof) to treat an immune-related disease, in combination with one or more other therapeutic agents. In certain embodiments, such a therapeutic agent works in combination (e.g., additively or synergistically) with the anti-CD3 antibody or fragment thereof to treat the immune-related condition. Therapeutic agents that can be administered in combination with an anti-CD3 antibody or fragment thereof include, but are not limited to, peptides, polypeptides, conjugates, nucleic acid molecules (e.g., DNA or RNA), small molecules, mimetic agents, synthetic drugs, inorganic molecules, and organic molecules. For example, a therapeutic agent can be an agent used to restore lost function (e.g., beta cell growth factors for diabetes), reduce inflammation (e.g., non-steroidal anti-inflammatory agents), or aid effect of the anti-CD3 antibody or fragment thereof (e.g., oral insulin). In some embodiments, stem cells or islet cells can be transplanted to restore lost function.

In certain embodiments, a therapeutic agent to be used in combination with an anti-CD3 antibody or antigen-binding fragment thereof is an immunomodulatory agent. In certain embodiments, such an immunomodulatory agent works in combination (e.g., additively or synergistically) with the anti-CD3 antibody or fragment thereof. Any of a variety of immunomodulatory agents known to those of skill in the art (e.g., those described above) may be administered in combination with an anti-CD3 antibody or fragment, as disclosed herein. Immunomodulatory agents typically affect one or more aspects of an immune response in a subject including, without limitation, an inflammatory response, a complement cascade, leukocyte and lymphocyte differentiation, proliferation, and/or effector function, monocyte and/or basophil counts, and the cellular communication among cells of the immune system. Non-limiting examples of additional immunomodulatory agents include proteinaceous agents such as cytokines, peptide mimetics, and antibodies (e.g., human, humanized, chimeric, monoclonal, polyclonal, Fvs, scFvs, Fab or F(ab′)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide). Other examples of immunomodulatory agents can be found, e.g., in United States Patent Publication Number 2005/0002934 A1 at paragraphs 259-275. In certain embodiments, an immunomodulatory agent is a chemotherapeutic agent. In certain embodiments, an immunomodulatory agent is an immunomodulatory agent other than a chemotherapeutic agent.

In certain embodiments, a therapeutic agent to be used in combination with an anti-CD3 antibody or antigen-binding fragment thereof for treating an immune-related disease is administered to a patient according to a different dosing regimen as the anti-CD3 antibody or fragment. For example, if a particular dosing regimen calls for an anti-CD3 antibody or fragment thereof to be administered to a patient on five consecutive days, a therapeutic agent may also be administered to the patient on only one day, or on two, three, four, six, seven, eight or more consecutive days, or on non-consecutive days. Those of ordinary skill in the art will be aware of suitable dosing regimens for a given therapeutic agent and will be able to administer such a therapeutic agent to a patient according to that therapeutic agent's effective dosing regimen.

Certain embodiments of methods and compositions provided herein are further illustrated by the following examples. The examples are provided for illustrative purposes only, and not to be construed as limiting the scope or content of the invention in any way.

### EXAMPLES

### Example 1

### Lower C-Peptide Secretion is Associated with Increased Blood Glucose Variability in Adults with New-Onset Type 1 Diabetes

To determine if increased blood glucose variability is a marker of reduced beta-cell function in adults with new-onset type 1 diabetes mellitus (NOT1DM), the relationship between glucose variability and C-peptide was evaluated in patients enrolled in DEFEND-1, a multinational placebo-controlled Phase 3 study of the safety and efficacy of an anti-CD3 monoclonal antibody (otelixizumab) in subjects with NOT1DM. Otelixizumab has been shown to preserve insulin secretion in a previous Phase 2 trial.

Seventy-two (72) subjects from DEFEND-1 were studied. Inclusion criteria, in brief, were as follows: Age 12-45 years, subjects 18-45 years are included in the current analysis; enrolled within 90 days of diagnosis with T1DM; BMI <32; Screening stimulated C-peptide level >0.20 and <3.50 nmol/L; Positive for at least one T1DM-associated autoantibody (GAD or IA2).

As part of the baseline assessments, participants had C-peptide levels measured during an MMTT performed according to a standard protocol. C-peptide secretion was measured at intervals for 120 minutes following Boost, and C-peptide area under the curve (AUC) was calculated. Also at baseline, subjects monitored blood glucose 7 times/day for 7 days.

Baseline data were examined for an association between beta-cell function (time-normalized C-peptide AUC over 120 minutes) and blood glucose variability (from self-monitoring). The study was approved by the ethics board associated with each site, and every subject provided written informed consent (Clinical trials identifier NCT00678886).

Two measurements of glucose variability were evaluated for their relationship to C-peptide: average daily risk range (ADRR) and mean amplitude of glycemic excursions (MAGE). ADRR and MAGE were calculated as described by Kovatchev et al. Diabetes Care, 29:2433-38 (2006); and Service et al., Diabetes, 19:644-655 (1970). Spearman correlations were used to assess associations among C-peptide AUC, HbA1c, MAGE, and ADRR in 72 subjects with sufficient data. Table 2 provides baseline characteristics of the 72 subjects.

TABLE 2
Baseline Characteristics of Subjects
(mean ± SD unless otherwise specified)

| | N = 72 | |
|---|---|---|
| Demographics | | |
| Age, years | 27.2 | (5.86) |
| Female, n (%) | 27 | (37.5) |
| Caucasian, n (%) | 71 | (98.6) |
| Clinical Characteristics | | |
| Time since diagnosis, days | 61.1 | (14.83) |
| BMI | 24.4 | (3.22) |
| Laboratory Parameters | | |
| HbA1c, % | 6.9 | (0.95) |
| C-peptide AUC, nmol/L/min | 0.94 | (0.536) |
| Fasting C-peptide, nmol/L | 0.31 | (0.210) |
| Max. Stim. C-peptide, nmol/L | 1.34 | (0.708) |

There were significant negative correlations between C-peptide AUC and both ADRR and MAGE (Table 3). Correlations between HbA1c and both measures of glucose variability were positive and significant.

TABLE 3
Spearman Correlation Coefficients

| | C-peptide AUC | HbA1c | MAGE | ADRR |
|---|---|---|---|---|
| C-peptide AUC | 1 | −0.15 | −0.40*** | −0.54*** |
| HbA1c | | 1 | 0.28* | 0.49*** |
| MAGE | | | 1 | 0.68*** |
| ADRR | | | | 1 |

***p < 0.001;
*p < 0.05

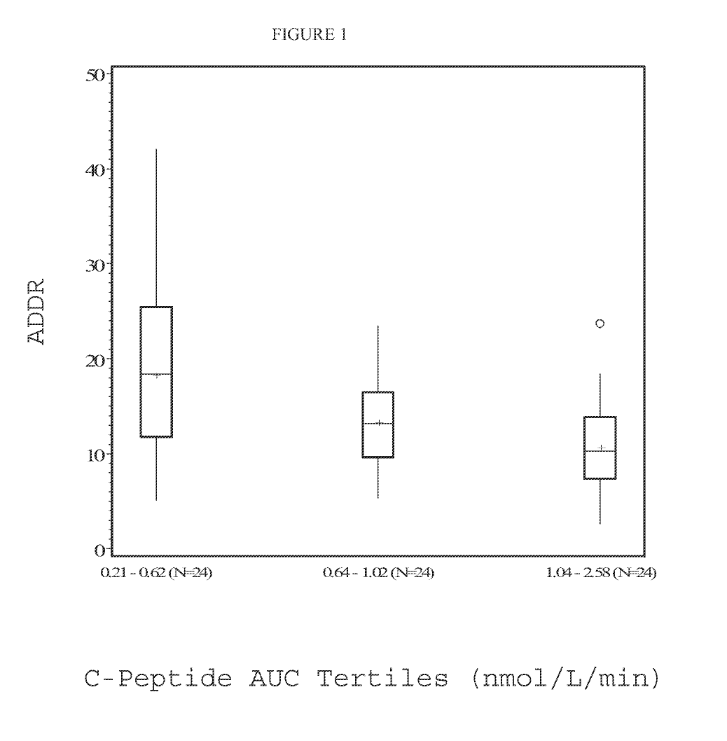

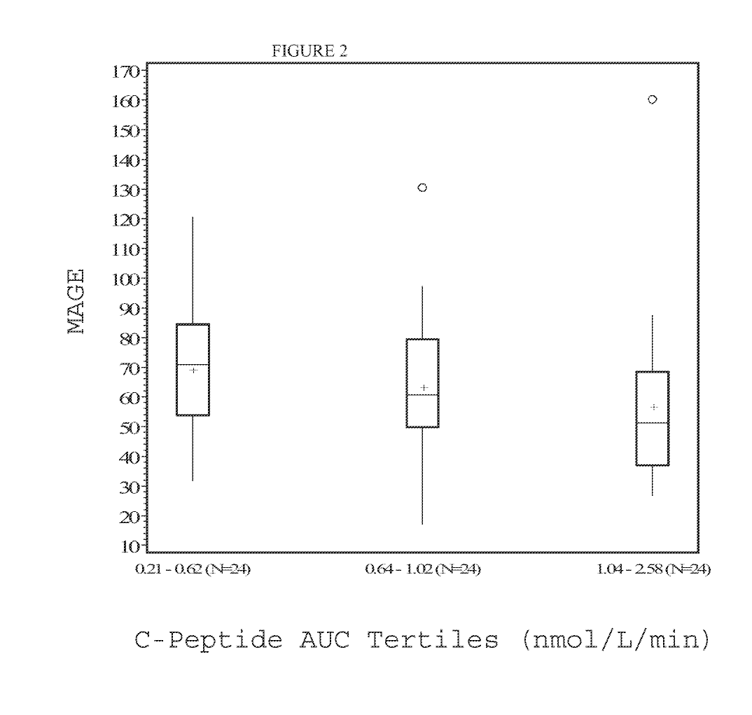

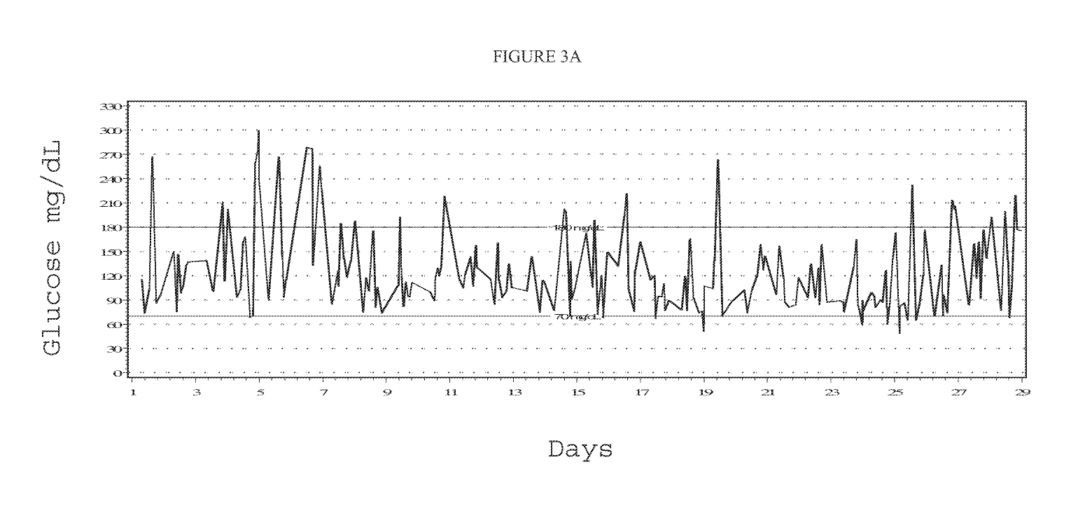

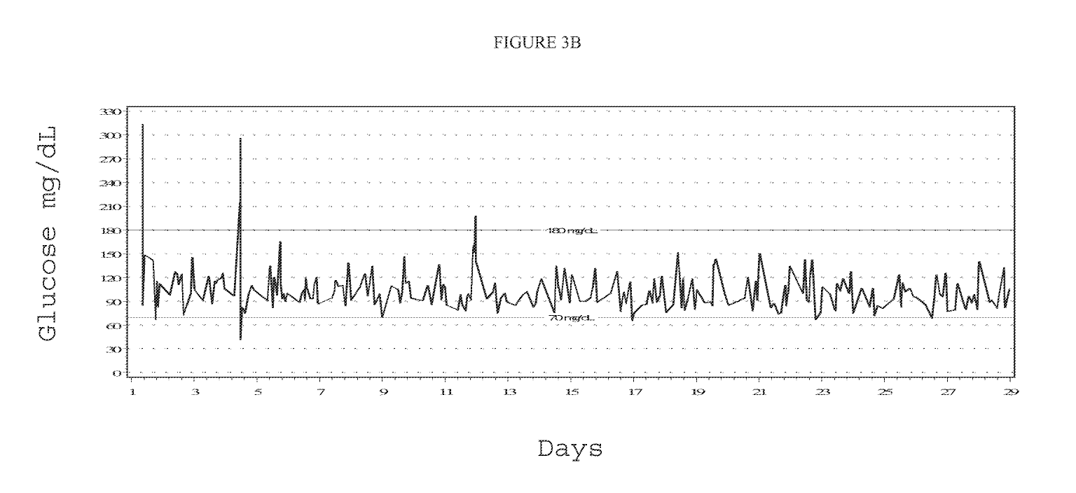

Subjects were divided into tertiles based on C-peptide AUC to further explore the association between C-peptide and other variables (e.g., MAGE, ADRR, and HbA1c). The results are presented in Table 4. FIG. 1 contains boxplot of ADRR by tertile of time-normalized simulated C-peptide AUC (nmol/L/min) FIG. 2 contains a boxplot of ADRR by tertile of time-nomalized simulated C-peptide AUC (nmol/L/min). In FIGS. 1 and 2, the box represents the 25th-75th percentile while circles represent values that are >1.5 times the interquartile range. FIG. 3 shows glucose levels from representative subjects. In FIG. 3A, the glucose levels of subjects representing tertile 1 (ADRR=19.3, C-peptide AUC=0.43 nmol/L/min) are shown over time. In FIG. 3B, the glucose levels of subjects representing tertile 3 (ADRR=10.0, C-peptide AUC=2.58 nmol/L/min) are shown over time.

TABLE 4
Variability by tertile of C-peptide (mean ± SD)

| | Tertile 1 | | Tertile 2 | | Tertile 3 | |
|---|---|---|---|---|---|---|
| | N = 24 | | N = 24 | | N = 24 | |
| C-peptide AUC | 0.21-0.62 | | 0.64-1.02 | | 1.04-2.58 | |
| range of tertile | | | | | | |
| MAGE | 69.9 | (22.93) | 64.0 | (24.62) | 57.5 | (28.66) |
| ADRR | 18.6 | (8.85) | 13.6 | (5.31) | 10.9 | (4.67) |
| HbA1c | 7.13 | (1.02) | 6.84 | (0.94) | 6.79 | (0.87) |

TABLE 5
Subjects Classified by ADRR According
to Risk of Future Hypoglycemia.

| | Tertile 1 | | Tertile 2 | | Tertile 3 | |
|---|---|---|---|---|---|---|
| Risk Category | N = 24 | | N = 24 | | N = 24 | |
| ADRR <20, low risk | 16 | (76%) | 21 | (88%) | 23 | (96%) |
| ADRR >20, med-high risk | 8 | (33%) | 3 | (12%) | 1 | (4%) |

In summary, blood glucose variability is associated with increased risk of long and short-term complications of diabetes. Some adults with NOT1DM have high blood glucose variability, which is related to reduced endogenous insulin secretion.

### Example 2

### Clinical Attributes of Adults with New-Onset Type 1 Diabetes

Patients with NOT1DM can continue to produce endogenous insulin after diagnosis for a variable period of time, commonly called the honeymoon period or the period of partial remission. Whether an individual is producing significant amounts of endogenous insulin is of interest to both patients and providers and can aid clinical decision making. The current gold standard for assessment of endogenous insulin production is C-peptide secretion in response to a mixed meal tolerance test (MMTT), but this is often not available in practice.

This example examines the clinical characteristics of adults with NOT1DM with high residual insulin secretion, as measured by C-peptide, and clinical parameters that are correlated with greater C-peptide secretion in response to an MMTT. It also was determined whether the insulin dose-adjusted HbA1c (IDAA1C) derived using data from patients <16 years, is useful for defining the honeymoon period in an adult population. An IDAA1C of <9 has been proposed as a clinical definition of honeymoon in a pediatric population. See Mortensen et al., Diabetes Care 32(8):1384-90 (2009) Epub 2009 May 12.

One hundred fifty-eight (158) subjects from DEFEND-1 were studied. Inclusion criteria, in brief, were as follows: Age 12-45 years, subjects 18-45 years are included in the current analysis; Enrolled within 90 days of diagnosis with T1DM; BMI <32; Screening stimulated C-peptide level >0.20 and <3.50 nmol/L; Positive for at least one T1DM-associated autoantibody (GAD, IA2, or ZnT8).

Subjects had C-peptide levels measured during an MMTT performed according to a standard protocol at baseline. C-peptide secretion was measured at intervals for 120 minutes and C-peptide area under the curve (AUC) was calculated. Subjects also kept an insulin diary for 7 consecutive days prior to the visit and had HbA1c measurements. Data from the baseline assessment (prior to administration of otelixizumab or placebo) was used to examine demographic and disease-related characteristics in subjects with high, medium, and low C-peptide secretion. The current analysis includes all adult subjects who participated in DEFEND-1 and who had available data. The study was approved by the ethics board associated with each site and every subject provided written informed consent.

Baseline C-peptide AUC, HbA1c, and insulin use data were analyzed. Daily insulin use was calculated as the mean of the subjects' total insulin use recordings (IU/kg) that were captured over a 7-day period within 14 days of an MMTT. The C-peptide AUC was calculated according to the trapezoidal rule and was normalized for time (i.e., AUC divided by 120 minutes). Insulin dose-adjusted HbA1c (IDAA1C) was calculated as HbA1c (%)+(4 X mean daily insulin use [IU/kg/day]). The baseline C-peptide AUC (one per subject) following an MMTT was classified as low, medium, or high. One-way analyses of variance (ANOVAs) were used to assess whether continuous parameters (i.e., all parameters except gender) differed among the C-peptide AUC groups (henceforth C-peptide groups). In addition, Spearman correlation coefficients were computed to evaluate the strength of relationships between C-peptide AUC and IDAAC, HbA1c, and mean daily insulin use. Table 6 provides baseline characteristics of the 158 subjects.

TABLE 6
Baseline Characteristics of Subjects
(mean ± SD unless otherwise specified)

| | N = 158 | |
|---|---|---|
| Demographics | | |
| Age, years | 27 | (5.7) |
| | Range 18-44 | |
| Female, n (%) | 64 | (40.5) |
| Caucasian, n (%) | 152 | (96.2%) |
| Clinical Characteristics | | |
| Time since diagnosis, days | 58 | (17.9) |
| BMI | 24 | (3.2) |
| Insulin Dose, U/kg | 0.35 | (0.20) |
| Laboratory Parameters | | |
| HbA1c, % | 7.2 | (1.26) |
| IDAA1C | 8.6 | (1.64) |
| C-peptide AUC, nmol/L/min | 0.86 | (0.483) |
| Fasting C-peptide, nmol/L | 0.3 | (0.22) |
| Max. Stim. C-peptide, nmol/L | 1.2 | (0.65) |

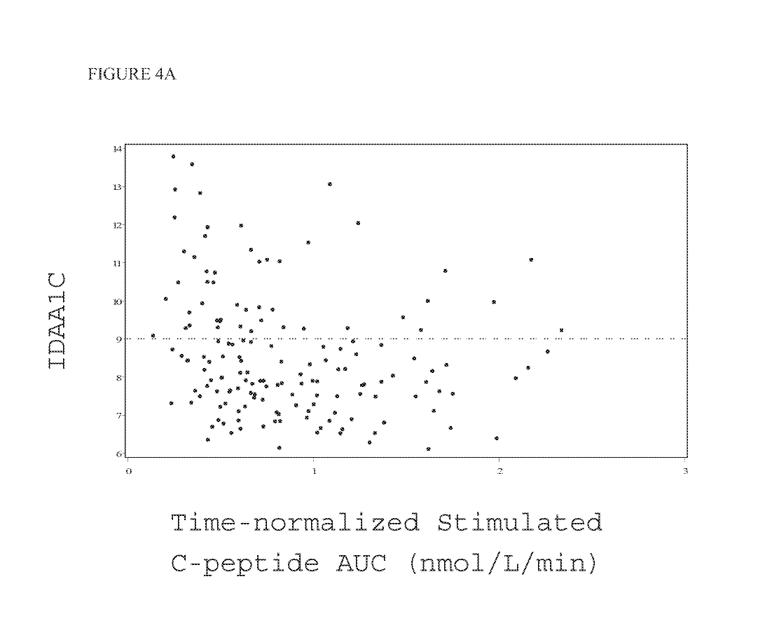

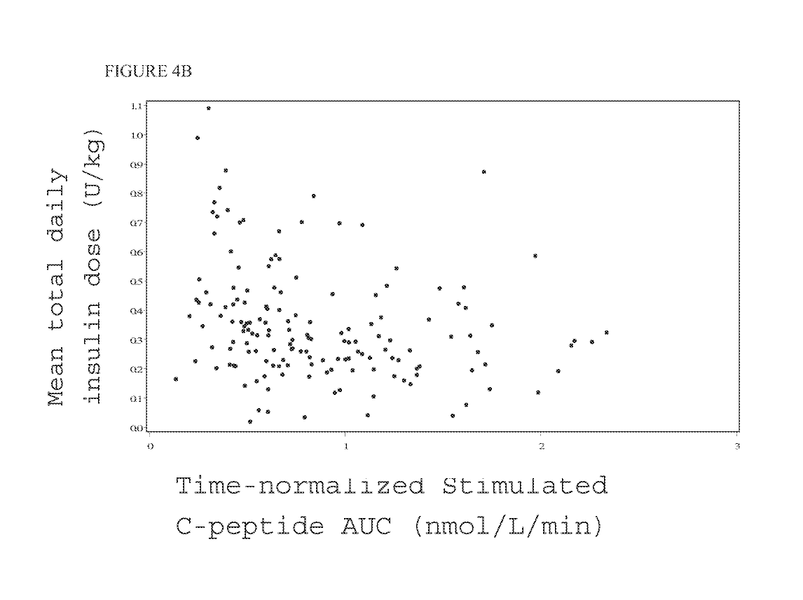

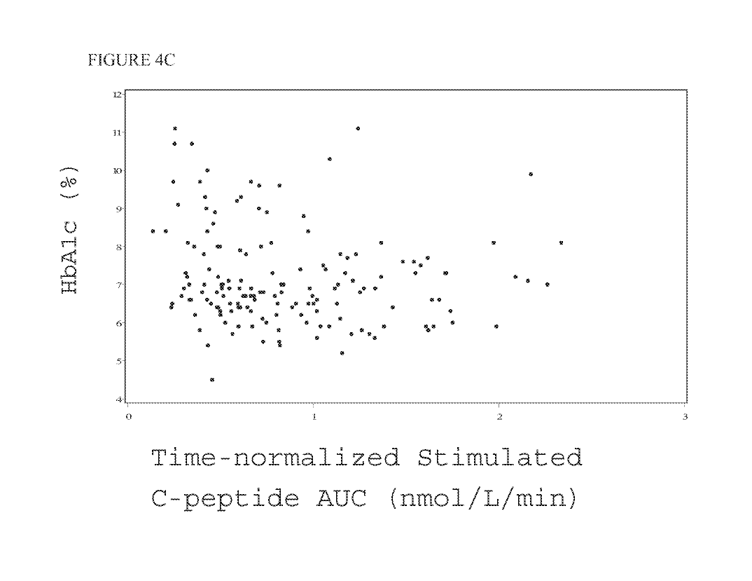

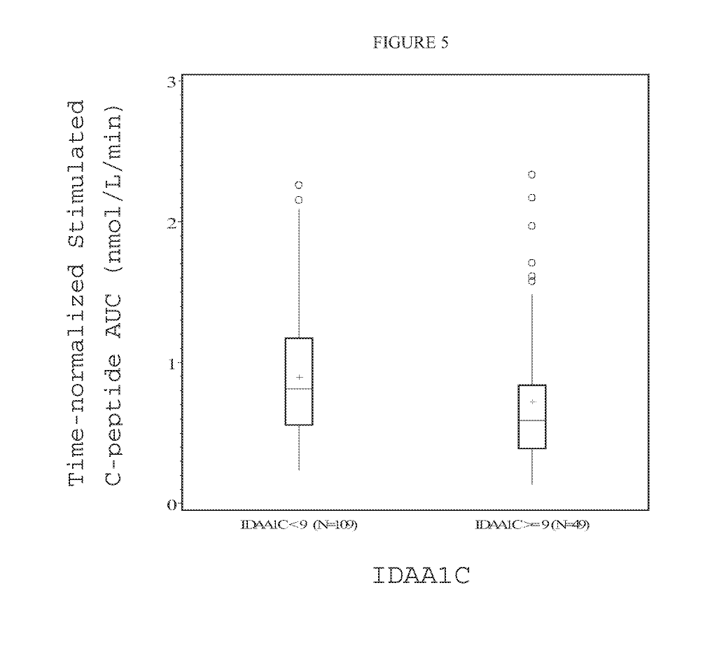

Data from 158 adult subjects were included (Table 6). The low, medium, and high C-peptide groups were defined as <0.5, 0.5 to 1.25, and >1.25 nmol/L/minute, respectively. Mean fasting and maximum stimulated C-peptide levels differed significantly among groups. Significant differences among groups also were found in mean HbA1c, daily insulin use, and IDAA1C (Table 7). See, FIGS. 4A-4C. Of the 109 subjects with an IDAA1C score of <9, 90 (83%) had a C-peptide AUC of >0.5 nmol/L/minute. See, FIG. 5. Spearman correlation demonstrated an inverse correlation between C-peptide AUC and IDAA1C, HbA1c, and daily insulin dose (r=−0.27, p<0.001; −0.16, p=0.04; −0.31, p<0.001).

TABLE 7
Subjects Grouped by Baseline C-peptide AUC (mean ± SD)

| | Low | | Medium | | High | | p-value |
|---|---|---|---|---|---|---|---|
| n | 42 | | 85 | | 31 | | |
| C-peptide | <0.5 | | 0.5-1.25 | | >1.25 | | |
| AUC Range, | | | | | | | |
| nmol/L | | | | | | | |
| Age, years | 27 | (5.5) | 26 | (5.5) | 26 | (6.5) | 0.21 |
| BMI | 24 | (2.6) | 24 | (3.25) | 25 | (3.8) | 0.07 |
| Female, | 13 | (31.0%) | 41 | (48.2%) | 10 | (32.3%) | 0.11 |
| n (%) | | | | | | | |
| Time since | 59 | (19.0) | 58 | (16.6) | 58 | (20.5) | 0.97 |
| diagnosis, | | | | | | | |
| days | | | | | | | |
| Fasting | 0.16 | (0.13) | 0.27 | (0.12) | 0.57 | (0.29) | <0.001 |
| C-peptide, | | | | | | | |
| nmol/L | | | | | | | |
| Max. Stim. | 0.53 | (0.15) | 1.17 | (0.34) | 2.24 | (0.40) | <0.001 |
| C-peptide, | | | | | | | |
| nmol/L | | | | | | | |
| HbA1c (%) | 7.7 | (1.51) | 7.0 | (1.16) | 6.9 | (0.95) | 0.007 |
| Insulin Dose, | 0.47 | (0.23) | 0.31 | (0.16) | 0.29 | (0.17) | <0.001 |
| U/kg | | | | | | | |
| IDAA1C | 9.5 | (1.93) | 8.3 | (1.43) | 8.1 | (1.27) | <0.001 |

Preserved beta cell function as defined on the basis of the Diabetes Control and Complications Trial (DCCT) is a fasting C-peptide of >0.08 nmol/L and a stimulated C-peptide of 0.2 nmol/L. By these criteria, even patients in the low C-peptide group of DEFEND-1 had preserved beta cell function within 90 days of diagnosis. Patients in the low C-peptide group had higher mean daily insulin requirements and HbA1c than patients in other groups. It should be noted that patients were studied on average 2 months post diagnosis so their mean daily insulin use and HbA1c should have reached steady state. An IDAA1C value of less than 9 was associated with higher C-peptide secretion but there was considerable overlap between groups. The IDAA1C is easily calculated and may be used in clinical practice to estimate C-peptide secretion when a C-peptide measurement is not available or practical.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A method of treating a patient suspected of having an immune-related disease, the method comprising (a) identifying a biomarker of said immune-related disease in said patient; (b) identifying a marker of pathogenic immunological activity in said patient; and (c) treating said patient with a therapeutically effective course of treatment if said biomarker of said immune-related disease and said marker of pathogenic immunological activity are identified in said patient, said course of treatment comprising a dosing regimen with an anti-CD3 antibody or antigen-binding fragment thereof, wherein said antibody or fragment does not bind or has reduced binding to at least one Fc (gamma) receptor compared to the IgG1 immunoglobulin molecule produced by the cell line ARH-77 deposited under ATCC catalog number CRL-1621.

3. (canceled)

4. A method of treating a patient suspected of having diabetes, the method comprising (a) identifying a biomarker of diabetes in said patient; (b) identifying a marker of pathogenic immunological activity in said patient; and (c) treating said patient with a therapeutically effective course of treatment if said biomarker of said immune-related disease and said marker of pathogenic immunological activity are identified in said patient, said course of treatment comprising a dosing regimen with an anti-CD3 antibody or antigen-binding fragment thereof, wherein said antibody or fragment does not bind or has reduced binding to at least one Fc (gamma) receptor compared to the IgG1 immunoglobulin molecule produced by the cell line ARH-77 deposited under ATCC catalog number CRL-1621.

5. (canceled)

6. A method of re-treating a patient with an immune-related disease, wherein said patient has undergone at least one course of treatment with a dosing regimen with an anti-CD3 antibody or antigen-binding fragment thereof after identification in said patient of a marker of said immune-related disease and a marker of pathogenic immunological activity, and wherein said antibody or fragment does not bind or has reduced binding to at least one Fc (gamma) receptor compared to the IgG1 immunoglobulin molecule produced by the cell line ARH-77 deposited under ATCC catalog number CRL-1621, said method comprising:
a) monitoring said patient for an indicator of return to active disease; and
b) re-dosing said patient with an additional course of treatment with a dosing regimen of said anti-CD3 antibody or antigen-binding fragment thereof if said indicator is identified in said patient.

7. (canceled)

8. (canceled)

9. The method of claim 4, wherein said marker of pathogenic immunological activity is pathogenic T cell activity.

10. The method of claim 4, wherein said marker of pathogenic immunological activity is autoimmune activity.

11. The method of claim 10, wherein said marker of autoimmune activity is autoimmune T cell activity.

12. The method of claim 4, wherein said patient has an immune-related disease.

13. The method of claim 12, wherein said immune-related disease is an autoimmune disease.

14. The method of claim 12, wherein said immune-related disease is diabetes.

15. The method of claim 14, wherein said immune-related disease is type 1 diabetes.

16. The method of claim 12, wherein said immune-related disease is selected from the group consisting of Crohn's disease, Graves' disease, Graves' ophthalmopathy, lupus erythematosus, multiple sclerosis, myasthenia gravis, psoriasis, psoriatic arthritis, rheumatoid arthritis, Sjögren's syndrome, systemic lupus erythematosus, and ulcerative colitis.

17. The method of claim 4, wherein said biomarker of said immune-related disease or said biomarker of diabetes is a biomarker of beta cell destruction.

18. The method of claim 17, wherein said biomarker of beta cell destruction is selected from the group consisting of amylin, glucagon, an islet-associated protein, and insulin production.

19. The method of claim 17, wherein said biomarker of beta cell destruction is glucose tolerance, glucose variability, insulin dose-adjusted HbA1c, or HbA1c.

20. The method of claim 18, wherein said biomarker of beta cell destruction is insulin production.

21.-66. (canceled)


SEQUENCE LISTING (10 sequences)

SEQ ID NO: 1 - protein, 449 aa, Artificial Sequence
  humanized murine antibody heavy chain
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, Artificial Sequence
  humanized murine antibody light chain
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 119 aa, Artificial Sequence [sequence omitted]

SEQ ID NO: 4 - protein, 111 aa, Artificial Sequence
  humanized murine antibody light chain variable region
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 5 - protein, 5 aa, Mus musculus
SFPMA

SEQ ID NO: 6 - protein, 17 aa, Mus musculus
TISTSGGRTYYRDSVKG

SEQ ID NO: 7 - protein, 10 aa, Mus musculus
FRQYSGGFDY

SEQ ID NO: 8 - protein, 13 aa, Mus musculus
TLSSGNIENNYVH

SEQ ID NO: 9 - protein, 7 aa, Mus musculus
DDDKRPD

SEQ ID NO: 10 - protein, 9 aa, Mus musculus
HSYVSSFNV